(19) United States
(12) Patent Application Publication
(10) Pub. No.: US 2014/0171371 A1
(43) Pub. Date: Jun. 19, 2014

(54) Compositions And Methods For The Diagnosis of Schizophrenia

(71) Applicant: The Trustees of Columbia University in the City of New York, New York, NY (US)

(72) Inventors: Vladimir Vacic, Brooklyn, NY (US); Shane McCarthy, Huntington, NY (US); Dheeraj Malhotra, San Diego, CA (US); Fiona Murray, San Diego, CA (US); Hsun-Hua Chou, San Diego, CA (US); Veronica Larach-Walters, Santiago (CL); Paul A. Insel, La Jolla, CA (US); Maria Karayiorgou, Riverdale, NY (US); Lynn E. DeLisi, Lincoln, MA (US); Jonathan Sebat, San Diego, CA (US)

(21) Appl. No.: 13/935,248

(22) Filed: Jul. 3, 2013

Related U.S. Application Data
(63) Continuation of application No. PCT/US2012/020683, filed on Jan. 9, 2012.
(60) Provisional application No. 61/430,883, filed on Jan. 7, 2011.

(51) Int. Cl. C12Q 1/68; G01N 33/74

(52) U.S. Cl. CPC C12Q 1/6883; G01N 33/74
USPC 514/13.1; 435/6.11; 506/9; 435/7.21; 506/16; 514/44 A

(57) ABSTRACT

The present disclosure relates to compositions and methods for the diagnosis of schizophrenia. In particular, the instant disclosure is directed to identification of novel copy number variants of sequences associated with the VIPR2 gene, including certain micro-duplications and triplications, and correlation of these copy number variants with schizophrenia.

### CROSS-REFERENCE TO RELATED APPLICATION

This application is a continuation of International Application No. PCT/US12/020683 filed Jan. 9, 2012, which claims the benefit of and priority to U.S. Provisional Application Ser. No. 61/430,883, filed Jan. 7, 2011, both of which are hereby incorporated by reference in their entireties, and to each of which priority is claimed.

### GRANT INFORMATION

This invention was made with government support under grant number NIH MH061399 awarded by the National Institutes of Health. The government has certain rights in the invention.

### 1. INTRODUCTION

The present disclosure relates to compositions and methods for the diagnosis of schizophrenia. In particular, the instant disclosure is directed to identification of novel copy number variants of sequences associated with the VIPR2 gene, including certain micro-duplications and triplications, and correlation of these copy number variants with schizophrenia.

### 2. BACKGROUND

Major progress has been made in understanding the genetic architecture of schizophrenia and other neuropsychiatric disorders. Studies have established the role of rare copy number variants (CNVs) in the etiology of schizophrenia²²⁻²⁷. Substantial risk for schizophrenia is conferred by large (>500 kb) CNVs at several loci, including microdeletions at 1q21.1²³, 3q29²⁸, 15q13.3²³ and 22q11.2²⁹ and microduplication at 16p11.2³⁰. All of the loci described above are hotspots for genomic rearrangement, where local segmental duplication architecture promotes frequent and nearly identical rearrangements by non-allelic homologous recombination (NAHR). Because of the high structural mutation rates at these loci, the strong phenotypic effects of the causal variants, and the excellent power of most array platforms to detect such large CNVs, these genomic hotspots were the first to be detected in studies of CNVs in schizophrenia. However, these CNVs collectively account for a small fraction (2-4%) of cases²⁶ and do not explain most of the increased CNV burden in schizophrenia reported in earlier studies^{22, 23}.

Since most of the genome lacks the duplication architecture of the NAHR hotspots described above and because a variety of mutational mechanisms can give rise to structural rearrangements, causal variants in other regions of the genome may consist of CNVs that are individually rarer and smaller (less than 500 KB) than those arising at NAHR hotspots. For example, microdeletions of the gene Neurexin-1 (NRXN1) are highly enriched in autism and schizophrenia³¹⁻³³ and consist of overlapping deletions with non-recurrent breakpoints. NRXN1 deletions are not flanked by segmental duplications, and may occur by different mutational mechanisms such as non-homologous end joining (NHEJ) or DNA replication-mediated rearrangement

In light of the fact that the aforementioned CNVs do not explain most of the increased CNV burden in schizophrenia, it would be desirable to identify new compositions and methods for facilitating the diagnosis of schizophrenia, particularly those that are associated with the identification of novel CNVs, such as those that are found outside of NAHR hotspots.

### 3. SUMMARY

The present disclosure relates to compositions and methods for the diagnosis of schizophrenia. In particular, the instant disclosure is directed to identification of novel copy number variants of sequences associated with the VIPR2 gene (VIPR2 CNV).

In certain embodiments, the VIPR2 CNV comprises a duplication or triplication of nucleic acid of chromosome 7.

In certain embodiments, the VIPR2 CNV comprises nucleic acid in region 7q36 of human chromosome 7.

In certain embodiments, the VIPR2 CNV comprises a region or segment of nucleic acid that is within 89 kb of the transcriptional start site of the VIPR2 gene.

In certain embodiments, the VIPR2 CNV comprises a region or segment of nucleic acid that is located, at least in part, in region 158,448,321-158,810,016; 158,731,401-158,810,016 or 158,448,321-158,605,936 of human chromosome 7, as described by the human genome build NCBI36/hg18 (produced by the International Human Genome Sequencing Consortium).

In certain embodiments, the VIPR2 CNV comprises a region or segment of exon 3 and/or exon 4 of the VIPR2 gene.

In certain embodiments, the VIPR2 CNV is associated with a second disorder or condition, for example, a psychiatric condition, disorder or phenotype. In certain embodiments, the VIPR2 CNV is associated with a pediatric neurodevelopment disorder. In certain embodiments, the VIPR2 CNV is associated with autism.

The present disclosure also relates to compositions and methods for the diagnosis of schizophrenia in a subject by detecting an increase in expression of a VIPR2 gene in a sample from the subject compared to VIPR2 expression in a sample from a control subject who does not have schizophrenia.

The present disclosure also relates to compositions and methods for the diagnosis of schizophrenia in a subject by detecting an increase in the level of cyclic-AMP in response to an agonist of VIPR2 such as, but not limited to, vasoactive intestinal peptide, or BAY55-9837, in a sample from the subject compared to the level of cyclic-AMP in a sample from a control subject who does not have schizophrenia. As one non-limiting example, the sample may comprise lymphoid or lymphoblastoid cells, which are then treated with the VIPR2 agonist prior to measurement of cAMP levels.

In certain embodiments, a VIPR2 CNV or VIPR2 expression product, can be detected through the use of, for example, the polymerase chain reaction (PCR), quantitative PCR, nucleic acid sequencing, nucleic acid microarray analysis or immunological detection.

In certain embodiments, the present disclosure provides for methods of treating a subject that has a VIPR2 CNV, an increased level of VIPR2 gene expression compared to a non-schizophrenic control, or an increased level of c-AMP in response to VIPR2 agonist compared to a non-schizophrenic control, wherein an agent is administered to the subject in an amount effective to decrease the level of VIPR2 in a sample from the subject.

In certain embodiments the agent inhibits the function of VIPR2 protein or reduces the level of functional VIPR2 protein present in a sample from the subject.

In certain embodiments, the agent is a VIPR2 protein antagonist or inhibitor.

In certain embodiments, the agent is an antisense molecule, RNAi molecule or siRNA molecule. In certain embodiments, the antisense, RNAi or siRNA molecule is complementary to a segment or region of a VIPR2 mRNA transcript. In certain embodiments, the antisense, RNAi or siRNA molecule hybridizes to and inhibits or reduces translation of VIPR2 mRNA.

In certain embodiments, the present disclosure provides for a kit for detecting at least one VIPR2 CNV, or VIPR2 expression product, wherein the kit comprises a plurality of oligonucleotide primers, each of which is capable of specifically hybridizing to genomic DNA associated with a VIPR2 CNV, or VIPR2 expression product, for example, genomic VIPR2 nucleic acid or VIPR2 mRNA.

In certain embodiments, the present disclosure provides for a kit for detecting VIPR2 expression, or cyclic-AMP level, wherein the kit comprises at least one antibody specific for a VIPR2 expression product, for example, VIPR2 protein, or cyclic-AMP, and a means of detecting the antibody when it is bound to the VIPR2 expression product or cyclic-AMP.

### 4. BRIEF DESCRIPTION OF THE FIGURES

FIG. 1A-1-1H Detection and validation of microduplications and triplications of 7q36.3. (a) Map of CNVs detected in the primary and secondary cohorts from the UCSC genome browser. (b) Plots of probe intensity ratios for 16 CNVs detected in the primary and MGS datasets. All are cases, with the exception of two controls which are indicated with an asterisk (*). Regions with estimated copy numbers of 2, 3 and 4 are highlighted in gray, blue and green respectively. Locations of four Sequenom validation assays are shown (dashed lines). (c-f) CNV genotypes were confirmed by MeZOD cluster plots of probe intensity ratios of the proximal and distal regions and in the primary dataset (c and d respectively) and secondary dataset (e and f respectively). Absolute copy numbers were confirmed for duplications and triplications of the proximal (g) and distal (h) regions by Sequenom MASSarray genotyping.

FIG. 2A-F Patterns of CNV inheritance in families. Pedigree diagrams are shown for families (a) LW102, (b) 02-016 and (e) 02-135, along with the Sequenom validation for families (d) LW102 and (e) 02-016 and (f) 02-135. Sequenom validation was performed on (a) mother and one of the affected sons and (b) all three family members, along with 10 CEU HapMap controls. Sequenom assays confirmed that duplications were present in the patients and maternally inherited from LW 102-2 and 02-0016-4.

FIG. 3A-D Duplications and Triplications of 7q36.3 result in increased VIPR2 transcription and cyclic-AMP signaling. (A) Quantitative PCR results of VIPR2 mRNA from lymphoblastoid cell lines. Two to four subjects were tested for each of four genotypes (subtelomeric duplication, VIPR2 duplication, exon 3/4 triplication, and normal diploid copy number as control). Results are expressed as the mean fold-change of CNV carriers relative to the mean of control samples. (B-C) Cyclic AMP accumulation was measured in the same cell lines in response to VIP (100 nM) (B) and the VPAC2 agonist BAY 55-9837 (100 nM) (C). Results are expressed as fold-change over forskolin/IBMX alone. (D) No significant differences were observed in cAMP response to another GPCR agonist, Prostaglandin E2 (PGE2, 1 μM); demonstrating that the effects are specific to VPAC2. For subjects, error bars represent standard error of the mean computed across replicates. Differences between the groups of 9 duplication carriers and 4 controls were tested using unpaired two sample t-test.

FIG. 4 Schematic representation of the two stage CNV association method. In stage 1, regions of interest (ROI) are defined as genomic loci recurrent in case samples and absent from controls. In stage 2, each ROI was split into segments based on breakpoints of overlapping CNVs and regions are investigated for association. Statistical significance of the ROI was based on the region with the minimal p-value (“association peak,” color coded red in the schematic), with appropriate permutation-based multiple testing correction.

FIGS. 5-1-5-3 Map of CNVs Detected in the 7q36.3 Region after Relaxing Stringent Filters on CNV Confidence Score and CNV Length. No additional CNVs were detected after eliminating the filter on the CNV confidence score. Additional small CNVs were detected in cases and controls after relaxing filters on CNV length (shown in green). The process of identifying of target regions in the primary cohort and testing association in the secondary cohort was repeated using the low-stringency CNV call set below. Target regions, as re-defined in the low-stringency call set, are shown in black. Association of target regions in the secondary cohort was statistically significant (permutation P-value=1.5*10−5, Peak OR=17.1 [3.3, Infinity]).

FIGS. 6-1-6-2 Fine Mapping of CNVs in MGS Subjects Using the NimbleGen HD2 Array. Fine mapping of 7q36.3 duplications was performed by re-scanning several individuals from the MGS study using the NimbleGen HD2 platform. NimbleGen results (right panel) were presented in FIG. 1b.

FIG. 7 Depicts tandem duplications of 7q36.3 confirmed in 2 patients by Fluorescence In Situ Hybridization (FISH).

FIGS. 8-1-8-7 Evaluating sensitivity of CNV detection in MGS cases and MGS controls based on concordance of segmentation calls with genotyping calls for 7 large copy number polymorphsisms (CNPs). For seven large (>100 Kb) CNPs reported in HapMap phase 3, clusters of median Z-scores were assigned genotypes as illustrated. The cluster that overlapped with a median Z-score of zero was assigned a genotype of “normal”, and distinct clusters where all Z-scores were greater than or less than 0 were assigned genotypes of “gain” or “loss”, respectively. Genotypes obtained in this manner were then compared with segmentation calls, and sensitivity was defined as the average fraction of genotyped gains or losses that were detected by segmentation per individual. By this approach, a reduced sensitivity to detect CNVs in controls was not observed (see Table 7).

FIG. 9 Gel electrophoresis of quantitative PCR (qPCR) products from lymphoblastoid cell line-derived RNAs analyzed in this study.

### SEQUENCE LISTING

The specification further incorporates by reference the Sequence Listing submitted via EFS on Jul. 3, 2013. Pursuant to 37 C.F.R. §1.52(e)(5), the Sequence Listing text file, identified as 0700504364ConSeqlist.txt, is 5,081 bytes and was created on Jul. 3, 2013. The Sequence Listing, electronically filed herewith, does not extend beyond the scope of the specification and thus does not contain new matter.

### 5. DETAILED DESCRIPTION

The present disclosure relates to compositions and methods for the diagnosis of schizophrenia. In particular, the instant application is directed to identification of novel copy number variants (CNV) of sequences associated with the VIPR2 gene, including certain micro-duplications and triplications, and correlation of these copy number variants with schizophrenia.

The present application is based at least in part on the identification of an association of CNVs of chromosome 7q36.3 with schizophrenia. For example, a genome-wide analysis of CNV in a primary cohort of 802 patients and 742 controls identified 114 genomic regions where multiple overlapping CNVs were detected exclusively in cases. These 114 regions were interrogated in a second series of 7,488 cases and 6,689 controls. Statistically significant associations were detected for copy number gains at 7q36.3 (P=4.0×10-5, OR=16.14 [3.06, ∞]). Microduplications with variable breakpoints occurred within a 362 kb region of 7q36.3, and were detected in 29 of 8,290 (0.35%) patients versus two of 7,431 (0.03%) controls in the combined sample (P=5.7×10−7, odds ratio (OR)=14.1 [3.5, 123.9]). All duplications at 7q36.3 overlapped or were located within 89 Kb upstream of the vasoactive intestinal peptide receptor VIPR2, and four patients carried partial triplications of this gene. VIPR2 transcription and cyclic-AMP signaling were significantly increased in cultured lymphocytes from patients with microduplications of 7q36.3.

For clarity and not by way of limitation, this detailed description is divided into the following sub-portions:

(i) VIPR2 CNVs;

(ii) Methods of Diagnosing Schizophrenia;

(iii) Methods of Treating Schizophrenia; and

(iv) Kits.

### 5.1 VIPR2 CNV

The present disclosure provides methods, compositions, and kits for diagnosing schizophrenia. Methods for detecting VIPR2 CNV are useful for detecting and/or diagnosing those who have or are at risk of developing schizophrenia. These methods and compositions will be useful in the diagnosis and treatment of persons with schizophrenia.

The VIPR2 gene encodes a Vasoactive Intestinal Peptide (VIP) Receptor 2 (VPAC2), a G protein-coupled receptor that is expressed in variety of tissues including, for example, brain, suprachiasmatic nucleus, hippocampus, amygdala, and hypothalamus. In certain embodiments, VIPR2 is a human VIPR2 gene, for example, a human VIPR2 gene described by GenBank accession number NM_{—}003382. In certain embodiments, the human VIPR2 gene encodes an amino acid sequence described by GenBank accession number NP_{—}003373.

In certain embodiments, VPAC2 binds VIP. In certain embodiments, binding of VPAC2 to VIP activates cyclic-AMP signaling and protein kinase A (PKA). In certain embodiments, VPAC2 functions to regulate synaptic transmission in the hippocampus. In certain embodiments, VPAC2 functions to promote proliferation of neural progenitor cells in the dentate gyms. In certain embodiments, activation of VPAC2 by VIP modulates learning and memory. In certain embodiments, VPAC2 also modulates circadian oscillations in the suprachiasmatic nucleus.

In certain embodiments, a “VIPR2 CNV” refers to a copy number variant (CNV) of a region or segment of nucleic acid of chromosome 7. In certain embodiments, the chromosome 7 is a human chromosome 7. In certain embodiments, the CNV comprises nucleic acid that is located on human chromosome 7 at position 7q36.3. In certain embodiments, the VIPR2 CNV comprises a subtelomeric region or segment of chromosome 7.

In certain embodiments, the VIPR2 CNV comprises a duplication of a region or segment of nucleic acid of chromosome 7.

In certain embodiments, the VIPR2 CNV comprises a triplication of a region or segment of nucleic acid of chromosome 7.

In certain embodiments, the VIPR2 CNV comprises a region or segment of an exon or intron of a VIPR2 gene.

In certain embodiments, the VIPR2 CNV comprises a region or segment of exon 3 of a VIPR2 gene.

In certain embodiments, the VIPR2 CNV comprises a region or segment of exon 4 of a VIPR2 gene.

In certain embodiments, the VIPR2 CNV comprises a region or segment of nucleic acid that is between about 1 and about 100 kb; or between about 1 and about 95 kb; or between about 1 and about 90 kb; or between about 1 and about 85 kb; or between about 1 and about 80 kb; or between about 1 and about 75 kb; or between about 1 and about 70 kb; or between about 1 and about 65 kb; or between about 1 and about 60 kb; or between about 1 and about 55 kb; or between about 1 and about 50 kb; or between about 1 and about 45 kb; or between about 1 and about 40 kb; or between about 1 and about 35 kb; or between about 1 and about 30 kb; or between about 1 and about 25 kb; or between about 1 and about 20 kb; or between about 1 and about 15 kb; or between about 1 and about 10 kb; or between about 1 and about 5 kb from the transcriptional start site of a VIPR2 gene.

In certain embodiments, the VIPR2 CNV comprises a region or segment of nucleic acid that is within 89 kb of the transcriptional start site of a VIPR2 gene.

In certain embodiments, the VIPR2 CNV comprises a region or segment of nucleic acid that is located in region 158,448,321-158,810,016 of human chromosome 7, as described by the human genome build NCBI36/hg18 (produced by the International Human Genome Sequencing Consortium).

In certain embodiments, the VIPR2 CNV comprises a region or segment of nucleic acid that is located in region 158,731,401-158,810,016 of human chromosome 7, as described by the human genome build NCBI36/hg18.

In certain embodiments, the VIPR2 CNV comprises a region or segment of nucleic acid that is located in region 158,448,321-158,605,936 of human chromosome 7, as described by the human genome build NCBI36/hg18.

In certain embodiments, the VIPR2 CNV comprises a region or segment of nucleic acid that is located in region 152,249,238-158,820,241; or 158,448,322-158,651,373; or 158,703,311-158,810,016; or 158,400,168-158,782,022; or 158,699,364-158,820,241; or 158,322,402-158,652,547; or 158,322,880-158,650,683; or 158,307,494-158,650,321; or 158,265,451-158,652,547; or 158,171,999-158,826,119; or 158,589,435-158,820,241; or 158,618,998-158,780,576; or 158,275,536-158,779,706; or 158,699,364-158,820,241; or 157,571,900-158,677,051; or 158,096,725-158,502,254; or 158,121,784-158,588,486; or 158,276,605-158,819,766; or 158,384,773-158,512,459; or 158,569,154-158,819,536; or 158,664,993-158,819,536; or 158,690,919-158,819,536; or 158,690,919-158,819,536; or 158,690,919-158,819,536; or 158,712,866-158,819,536; or 158,712,866-158,819,536; or 158,712,866-158,819,536; or 158,719,441-158,819,536; or 158,719,441-158,819,536; or 158,543,969-158,614,169; or 158,540,185-158,621,330; or 158,396,852-158,456,333; or 158,562,524-158,614,169; or 158,550,023-158,614,169; or 158,654,446-158,726,336 of human chromosome 7, as described by the human genome build NCBI36/hg18.

### 5.2 Methods of Diagnosing Schizophrenia

According to the present disclosure, VIPR2 CNVs are present in individuals with schizophrenia, or at risk of developing schizophrenia or schizoaffective disorder. Detection of these VIPR2 CNVs can be used to diagnose schizophrenia or schizoaffective disorder in a subject and can be used in conjunction with other criteria, such as those set forth in the Diagnostic and Statistical Manual of Mental Disorders IV (DSMIV) to support a clinical psychiatric diagnosis of schizophrenia or schizoaffective disorder. Detection of a VIPR2 CNV is also helpful to identify any adverse health effects associated therewith, and thus detection can be useful for finding and treatment of schizophrenia. While the present disclosure is exemplified in humans, its extension to other species including mammals is contemplated. Assays such as RT-PCR, PCR, qPCR, DNA and RNA sequencing, microarray analysis and any other genome-based analyses known in the art, along with any immunoassays known in the art, may be used to detect a VIPR2 CNV in a sample. In addition, such analyses may be qualitative or quantitative.

According to the present disclosure, a “subject” or “patient” is a human or non-human animal. Although the animal subject is preferably a human, the concepts, compounds and compositions of the disclosure have application in veterinary medicine as well, e.g., for the treatment of domesticated species, farm animal species, and wild animals or zoological garden animals.

In humans, the VIPR2 CNVs disclosed herein may be detected individually or in combination to provide a diagnostic evaluation of schizophrenia. Other VIPR2 CNVs from other species may prove useful, alone or in combination, for similar purposes.

In certain embodiments, the present disclosure provides for methods of diagnosing schizophrenia in a subject comprising detecting an increased level of VIPR2 expression in a sample from the subject compared to the level of VIPR2 expression in a sample from a control subject that does not have schizophrenia. In certain embodiments, the level of expression of VIPR2 is the level of transcription of VIPR2 in the samples, for example, as determined by the level of VIPR2 mRNA. In certain embodiments, the level of expression of VIPR2 is the level of VIPR2 protein detected in the samples, for example, the level of Vasoactive Intestinal Peptide Receptor 2 (VPAC2) protein detected in the samples.

In certain embodiments, the present disclosure provides for methods of diagnosing schizophrenia in a subject by detecting an increased level of cyclic-AMP activation, signaling or accumulation in a sample from the subject when the sample is contacted with a VPAC2 agonist, for example, VIP or BAY55-9837, compared to the level of cyclic-AMP activation, signaling or accumulation in a sample from a control subject that is contacted with a VPAC2 agonist, wherein the control subject does not have schizophrenia. As one non-limiting example, the sample of the subject may comprise lymphoid or lymphoblastic cells from the subject, which are treated with VPAC2 agonist, and then the consequent change in cAMP level may be measured.

In certain embodiments, the samples described herein can be derived from any tissues, cells and/or cells in biological fluids from, for example, a mammal or human to be tested.

In certain embodiments, a VIPR2 CNV is associated with a second disorder or condition, for example, a psychiatric condition, disorder or phenotype. In certain embodiments, the VIPR2 CNV is associated with a pediatric neurodevelopment disorder. In certain embodiments, the VIPR2 CNV is associated with autism As used herein, the terms “associated with a second disorder” mean that the VIPR2 CNV and a second disorder (i.e., a disorder other than schizophrenia), exhibit a degree of linkage, wherein the VIPR2 CNV and the second disorder are present together in an individual at a higher frequency than if their occurrences were independent of each other.

The present disclosure encompasses nucleic acid segments that are complementary, or essentially complementary, to the nucleic acid regions or segments of chromosome 7 described herein, for example, the VIPR2 gene and its chromosomal region, for example 89 kb upstream of the transcriptional start site. Nucleic acid sequences that are “complementary” are those that are capable of base-pairing according to the standard Watson-Crick complementary rules. As used herein, the term “complementary sequences” means nucleic acid sequences that are substantially complementary, as defined as being capable of hybridizing to a specified nucleic acid segment, under relatively stringent conditions as known in the art. Such sequences may encode the entire VIPR2 protein product encompassed herein or functional or non-functional fragments thereof.

A nucleic acid may be contained in a host cell, in some cases, capable of expressing the product of that nucleic acid. In addition to diagnostic and therapeutic considerations, cells expressing nucleic acids of the present disclosure may prove useful in the context of screening for agents that induce, repress, inhibit, augment, interfere with, block, abrogate, stimulate or enhance the expression, distribution, turnover, or detectability of VIPR2 CNVs.

Hybridizing segments may be relatively short nucleic acids, often termed oligonucleotides. Sequences of at least 10 bases long, for example, sequences of at least 17 or at least 22 bases long, should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of any number from 8 to 100 or more base pairs will be used, although others are contemplated. Longer polynucleotides are contemplated as well. Such oligonucleotides will find use, for example, as probes in Southern and Northern blots and as primers in amplification reactions.

Suitable hybridization conditions will be well known to those of skill in the art. Accordingly, the nucleotide sequences of the disclosure may be used for their ability to selectively form duplex molecules with complementary stretches of DNA or RNA fragments. Depending on the application envisioned, one will desire to employ varying conditions of hybridization to achieve varying degrees of selectivity of probe towards target sequence. For applications requiring high selectivity, one will typically desire to employ relatively stringent conditions to form the hybrids, e.g., one will select relatively low salt and/or high temperature conditions, such as provided by about 0.02 M to about 0.15 M NaCl at temperatures of about 50° C. to about 70° C. Such selective conditions tolerate little, if any, mismatch between the probe and the template or target strand, and would be particularly suitable for isolating protein-encoding DNA segments. Detection of DNA segments via hybridization is well-known to those of skill in the art, and the teachings of U.S. Pat. Nos. 4,965,188 and 5,176,995 (each specifically incorporated herein by reference) are exemplary of the methods of hybridization analyses.

In certain applications, lower stringency conditions are required. Under these conditions, hybridization may occur even though the sequences of probe and target strand are not perfectly complementary, with mismatches at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature.

In certain embodiments, it will be advantageous to employ nucleic acid sequences of the present disclosure in combination with an appropriate means, such as a label, for determining hybridization. A wide variety of appropriate indicator means are known in the art, including fluorescent, radioactive, enzymatic or other ligands, such as avidin/biotin, which are capable of giving a detectable signal. In certain embodiments, one will likely desire to employ a fluorescent label or an enzyme tag, such as urease, alkaline phosphatase, luciferase, or peroxidase, instead of radioactive or other environmental undesirable reagents. In the case of enzyme tags, colorimetric indicator substrates are known that can be employed to provide a means visible to the human eye or spectrophotometrically, to identify specific hybridization with complementary nucleic acid-containing samples.

In general, it is envisioned that the hybridization probes described herein will be useful both as reagents in solution hybridization as well as in embodiments employing a solid phase. In embodiments involving a solid phase, the test DNA (or RNA) is adsorbed or otherwise affixed to a selected matrix or surface. This fixed, single-stranded nucleic acid is then subjected to specific hybridization with selected probes under desired conditions. The selected conditions will depend on the particular circumstances based on the particular criteria required (depending, for example, on the G+C content, type of target nucleic acid, source of nucleic acid, size of hybridization probe, etc.). Following washing of the hybridized surface so as to remove nonspecifically bound probe molecules, specific hybridization is detected, or even quantitated, by means of the label.

Probes and primers of the present disclosure are useful for PCR, qPCR, nucleic acid sequencing, microarray analysis, site-directed, and site-specific mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

### Detection of VIPR2 CNV or VIPR2 Expression Product Using Nucleic Acid Microarrays

In certain embodiments of the disclosure, the presence of a VIPR2 CNV or VIPR2 expression product in a sample can be determined by using nucleic acid microarrays, or gene chip technology (see, e.g., U.S. Pat. No. 7,455,975). As used herein, a “microarray” is an array of distinct polynucleotides, oligonucleotides, polypeptides, peptides, or antibodies affixed to a substrate, such as paper, nylon, or other type of membrane; filter; chip; glass slide; silicone or any other type of suitable support. The use of microarray technology for detecting VIPR2 CNV is further described in the embodiments disclosed in the Examples of the present application.

In certain embodiments, the microarray technology involves the positioning of highly condensed and ordered arrays of nucleic acid probes, for example, DNA oligonucleotides, on a substrate, for example, a glass slide or nylon membrane. Each oligonucleotide may comprise a nucleotide sequence that is complementary to a portion of, for example, a VIPR2 CNV or VIPR2 expression product, wherein the oligonucleotide can be placed, for example, on a single glass slide or nylon membrane. The resulting microarrays can then be used to screen for the presence of a VIPR2 CNV or VIPR2 expression product expressed in a sample to be screened.

In certain embodiments, a nucleic acid microarray may be utilized by preparing labeled nucleic acid from a sample to be screened, and hybridizing such labeled nucleic acid with the array. In addition, labeled nucleic acid of a designated control sequences may be prepared (or in the event that the array is sold as part of a kit, could be supplied to the user). Radioactive, colorimetric, chemiluminescent or fluorescent tags may be used for labeling of nucleic acid sequences from the sample and for the control. Numerous techniques for scanning arrays, detecting fluorescent, chemiluminescent, or colorimetric output, are known in the art and may be used for detecting hybridization of a nucleic acid from a test sample to the microarray. For example, a high-throughput fluorescent microarray scanning system (ScanArray®, PerkinElmer Life And Analytical Sciences, Inc., Waltham, Mass., USA), or a colorimetric microarray scanner (ArrayIt® SpotWare™, TeleChem International, Inc., Sunnyvale, Calif., USA) can be used. Additional microarray systems that can be used according to the methods of the present disclosure include the NimbleGen platform (Roche NimbleGen, Inc., Madison, Wis.), GeneChip® Human Mapping Array Sets (Affymetrix, Inc., Santa Clara, Calif.) and Genome-Wide Human SNP Arrays (Affymetrix, Inc., Santa Clara, Calif.).

The present disclosure contemplates the preparation of one or more specialized microarrays (e.g., oligonucleotide microarrays or cDNA microarrays) comprising one or more polynucleotides encoding one or more VIPR2 CNV, VIPR2 nucleic acid sequence or complementary sequences, or fragments thereof. In accordance with this aspect of the disclosure, the oligonucleotide sequences or cDNA sequences include any of the disclosed VIPR2 CNV or VIPR2 polynucleotides or fragments or combinations thereof, and are contained on a microarray, e.g., a oligonucleotide microarray or cDNA microarray in association with, or introduced onto, any supporting materials, such as glass slides, nylon membrane filters, glass or polymer beads, or other types of suitable substrate material.

Methods for producing and using DNA microarrays are well known in the art (see, e.g. Rampal J. B., 2001, DNA Arrays: Methods and Protocols, Humana Press, Inc., Totowa, N.J.; Schena M., 2000, Microarray Biochip Technology, Eaton Publishing; and Schena M., 2002, Microarray Analysis, John Wiley & Sons). For example, to determine gene expression using microarray technology, polynucleotides, e.g., RNA, DNA, or cDNA, are isolated from a biological sample, e.g., cells expressing a VIPR2. The isolated nucleic acid is detectably labeled, e.g., by fluorescent, enzyme, or chemiluminescent label, and applied to a microarray, e.g., one or more nucleic acid microarrays provided by this disclosure which comprises, for example, oligonucleotides complimentary to the labeled cellular derived nucleic acid applied to the microarray. The array is then washed to remove unbound material and visualized by staining or fluorescence, or other means known in the art depending on the type of label utilized.

For the purpose of example, and not limitation, microarrays of the disclosure may be prepared by amplifying VIPR2 CNVs and/or VIPR2 cDNAs or fragments thereof by PCR or RT-PCR, and arraying the PCR products from a microtiter plate onto silyated microscope slides using high-speed robotics. Printed arrays may be incubated in a humid chamber to allow rehydration of the array elements and rinsed, in, for example, 0.2% SDS, water, and sodium borohydride solutions.

Further, a commercially available microarray and/or commercially available software may be used to detect VIPR2 CNV according to the invention (see, for instance, the working example below). Non-limiting examples of microarrays which may be used to detect a VIPR2 CNV include microarrays and associated software marketed by Affymetrix, such as Genome-wide Human SNP Array 6 and the associated Genotyping Console (e.g., version 4.1.1, prior or subsequent versions), or Illumina, such as Whole Genome Genotyping and Copy Number Variation Analysis, such as the Omni Family of Microarrays, and associated software.

### Detection of VIPR2 CNV or VIPR2 Expression Product Using Fluorescent In Situ Hybridization

In certain embodiments, the present disclosure provides methods for the detection of a VIPR2 CNV or VIPR2 expression product comprising the use of Fluorescence in situ Hybridization (FISH). The term “in situ hybridization” generally refers to hybridization of a nucleic acid probe to a nucleic acid target that is part of a cytological or histological preparation. Typically, FISH methods involve the following steps: (a) fixing the tissue or other biological material under investigation to a support (e.g., glass slide or wall of a micro titer well), (b) treatment of the tissue or material to increase accessibility of FISH probe to target nucleic acid, (c) contacting the tissue or material containing the target nucleic acid with probes to form specific hybridization complexes, (d) post hybridization washes of the complexes to selectively remove probes that are not specifically hybridized to the target, and (e) detection of probes that have formed hybridization complexes with target nucleic acid molecules. Such methods are described in a number of sources, including: Gall and Pardue, (1981) Methods of Enzymology 21:470-480; Henderson, (1982) International Review of Cytology, 76:1-46; and Angerer, et al., (1985) in Genetic Engineering: Principles and Methods (Setlow and Hollaender, Eds.) vol. 7, pp. 43-65, Plenum Press, New York.

### PCR Detection of VIPR2 CNV

In certain embodiments of the present disclosure, the presence of VIPR2 CNVs can be detected by polymerase chain reaction (PCR).

In certain embodiments, the VIPR2 CNV is amplified from a genomic DNA sample of a subject. Any one or more of the VIPR2 CNVs disclosed herein can be amplified through PCR by using at least one set of primers for each VIPR2 CNV. Following PCR amplification, the PCR amplification products can be sequenced using standard techniques known in the art, and the sequence can be compared to the sequence of a control sample, or to the corresponding wild type sequence.

In certain embodiments, the presence of the VIPR2 CNV can be determined by detecting a difference in size between the subject sample PCR product and the control sample PCR product. Such differences in size can be determined, for example, by gel electrophoresis or any other method known in the art for detecting the size of a PCR product.

### Quantitative RT-PCR

According to the present disclosure, the expression level of a VIPR2 gene can be detected through the use of quantitative polymerase chain reaction (qPCR), or quantitative reverse transcriptase-polymerase chain reaction (qRT-PCR), which utilizes competitive techniques employing an internal homologous control that differs in size from the target, for example, by a small insertion or deletion. Non-competitive and kinetic quantitative PCR or RT-PCR may also be used. Experiments may combine real-time, kinetic PCR or RT-PCR detection together with an internal homologous control that can be simultaneously detected alongside the target sequences. In certain embodiments, real time quantitative PCR may provide the capability of measuring the level of VIPR2 gene product amplified through PCR. In certain embodiments, quantitative PCR may require only a nominal amount of a sample to perform such experiments.

Quantitative amplification is based on the monitoring of a signal (e.g., fluorescence of a probe) representing copies of a template in cycles of an amplification (e.g., PCR) reaction. In the initial cycles of the PCR, a very low signal is observed because the quantity of the amplification product formed does not support a measurable signal output from the assay. After the initial cycles, as the amount of formed amplification product increases, the signal intensity increases to a measurable level and reaches a plateau in later cycles when the PCR enters into a non-logarithmic phase. Through a plot of the signal intensity versus the cycle number, the specific cycle at which a measurable signal is obtained from the PCR reaction can be deduced and used to back-calculate the quantity of the target before the start of the PCR. The number of the specific cycles that is determined by this method is typically referred to as the cycle threshold (Ct). Exemplary methods are described in, e.g., U.S. Pat. Nos. 6,180,349; 6,033,854; and 5,972,602.

In certain embodiments, a method for detection of amplification products is, for example, the 5′-3′ exonuclease activity during PCR reaction (also referred to as the TaqMan™ assay) (see, e.g., U.S. Pat. Nos. 5,210,015 and 5,487,972). This assay detects the accumulation of a specific PCR product by hybridization and cleavage of a doubly labeled fluorogenic probe (the “TaqMan™” probe) during the amplification reaction. The fluorogenic probe consists of an oligonucleotide labeled with both a fluorescent reporter dye and a quencher dye. During PCR, this probe is cleaved by the 5′-exonuclease activity of DNA polymerase if it hybridizes to the segment being amplified. Cleavage of the probe generates an increase in the fluorescence intensity of the reporter dye.

In certain embodiments, detection of amplification products may utilize, by way of example, and not by way of limitation, energy transfer according to the “beacon probe” method described by Tyagi and Kramer (Nature Biotech. 1996; 14:303-309.), and disclosed by U.S. Pat. Nos. 5,119,801 and 5,312,728. This method employs oligonucleotide hybridization probes that can form hairpin structures. On one end of the hybridization probe (either the 5′ or 3′ end), there is a donor fluorophore, and on the other end, an acceptor moiety. In the Tyagi and Kramer method, the acceptor moiety is a quencher, wherein the acceptor absorbs energy released by the donor, but then does not itself fluoresce. Thus, when the beacon is in the open conformation, the fluorescence of the donor fluorophore is detectable, whereas when the beacon is in hairpin (closed) conformation, the fluorescence of the donor fluorophore is quenched. When employed in PCR, the molecular beacon probe, which hybridizes to one of the strands of the PCR product, is in the open conformation and fluorescence is detected, while those that remain unhybridized will not fluoresce (Tyagi and Kramer, 1996). As a result, the amount of fluorescence will increase as the amount of PCR product increases, and thus may be used as a measure of the progress of the PCR. Those of skill in the art will recognize that other methods of quantitative amplification are also available.

Various other techniques for performing quantitative amplification of nucleic acids are also known. For example, some methodologies employ one or more probe oligonucleotides that are structured such that a change in fluorescence is generated when the oligonucleotide(s) is hybridized to a target nucleic acid. For example, one such method involves is a dual fluorophore approach that exploits fluorescence resonance energy transfer (FRET), e.g., LightCycler™ hybridization probes, where two oligo probes anneal to the amplification product. The oligonucleotides are designed to hybridize in a head-to-tail orientation with the fluorophores separated at a distance that is compatible with efficient energy transfer. Other examples of labeled oligonucleotides that are structured to emit a signal when bound to a nucleic acid or incorporated into an extension product include: Scorpions™ probes (e.g., Whitcombe et al., Nature Biotechnology 1999; 17:804-807, and U.S. Pat. No. 6,326,145), Sunrise™ (or Amplifluor™) probes (e.g., Nazarenko et al., Nuc. Acids Res. 1997; 25:2516-2521, and U.S. Pat. No. 6,117,635), and probes that form a secondary structure that results in reduced signal without a quencher and that emits increased signal when hybridized to a target (e.g., Lux Probes™).

Quantitation of a specific amplified product at the end of an amplification reaction (i.e., end-point PCR) can be employed to quantify the sequences in the final amplified population that match the sequence of DNA which remained undigested by a restriction enzyme. The end-point PCR analysis may be employed under conditions in which the reaction can be analyzed before the reactant nears depletion for a quantitative comparison. Most typically this is done through a comparison of reaction products following a limited number of cycles. For example, a reaction is allowed to cycle 10 times, 15 times, 20 times or 30 times. The quantities of end point PCR products can be compared to each other and an analysis of sequences from the differential enzyme treatments of the DNA sample can be made.

### Detection of VIPR2 CNV Using Nucleic Acid Sequencing

In certain embodiments, the presence of a VIPR2 CNV in a subject or control sample can be determined through sequencing (i.e. determining the nucleotide order of a given DNA or RNA fragment) of a genomic DNA product present in the sample. Any sequencing methods known in the art may be used to determine the nucleotide order of the VIPR2 CNV DNA or RNA.

For example, and not by way of limitation, chain terminator sequencing (i.e. Sanger sequencing) may be used to sequence the VIPR2 CNV, wherein extension of a polynucleotide is initiated at a specific site on the template VIPR2 CNV nucleic acid (e.g., DNA) by using a short oligonucleotide “primer” complementary to the template at that region. The classical chain-termination method requires a single-stranded DNA template, a DNA primer, a DNA polymerase, radioactively or fluorescently labeled nucleotides, and modified nucleotides that terminate DNA strand elongation (e.g., dideoxynucleotides). The DNA sample may be divided into four separate sequencing reactions, containing all four of the standard deoxynucleotides (dATP, dGTP, dCTP and dTTP) and the DNA polymerase. One of the four dideoxynucleotides (ddATP, ddGTP, ddCTP, or ddTTP) are added to each of the four reactions, which are the chain-terminating nucleotides, lacking a 3′-OH group required for the formation of a phosphodiester bond between two nucleotides, thus terminating DNA strand extension and resulting in various DNA fragments of varying length.

Newly synthesized and labeled DNA fragments are heat denatured, and separated by size by, for example, gel electrophoresis, with each of the four reactions run in one of four individual lanes of the gel (lanes A, T, G, C). The DNA bands may be visualized by autoradiography or UV light, and the DNA sequence can be directly read off the X-ray film or gel image.

In one embodiment, the primer is labeled (e.g., a fluorescent or radioactive label). In other embodiments, the chain-terminator nucleotides are labeled, for example, in ‘dye terminator sequencing’. In dye terminator sequencing, complete sequencing may be performed in a single reaction, wherein each of the di-deoxynucleotide chain-terminators (e.g., ddATP, ddGTP, ddCTP, and ddTTP) are labeled with a separate fluorescent dye which fluoresces at a different wavelength. The sequence of the template may be determined by separating the synthesized polynucleotide by size and determining the order of the dye signals exhibited by the reaction products.

In certain embodiments, sequencing may be performed according to the “pyrosequencing” method as described in, for example, Ronaghi et al., Analytical Biochemistry 1996; 242(1):84-9; Ronaghi et al, Science 1998; 281:363-365; and Nyrén, Methods Mol Biology 2007; 373: 1-14. Pyrosequencing is a nucleic acid (e.g., DNA) sequencing technique that relies on detection of pyrophosphate release upon nucleotide incorporation rather than chain termination with dideoxynucleotides. Thus, detection of the nucleotide order of the polynucleotide synthesized in the synthesis reaction may be determined in real time as the polynucleotide is extended.

The sequencing of a nucleic acid sample (i.e. determining the nucleotide order of a given DNA or RNA fragment) is not limited to any one technique. The present disclosure contemplates the use of any sequencing technique known in the art and, for example, new sequencing techniques arising in the future of the sequencing art.

Immunologic Detection of VIPR2 Protein and c-AMP

In certain embodiments, the present disclosure entails the use of antibodies in the immunologic detection of VIPR2 protein or c-AMP present in a subject or control sample. According to the present disclosure, such immunological detection can be used to detect and quantify the amount of VIPR2 protein or c-AMP present in the subject or control samples. Various useful immunodetection methods have been described in the scientific literature, such as, e.g., Nakamura et al. Handbook of Experimental Immunology (4th Ed.), Weir, E., Herzenberg, L. A., Blackwell, C., Herzenberg, L. (Eds.), Vol. 1, Chapter 27, Blackwell Scientific Publ., Oxford, 1987. Immunoassays, in their most simple and direct sense, are binding assays. Certain immunoassays include, but are not limited to, enzyme linked immunosorbent assays (ELISAs), Western blots and radioimmunoassays (RIA). Immunohistochemical detection using tissue sections also is particularly useful. However, it will be readily appreciated that detection is not limited to such techniques. For example, Western blotting, dot blotting, FACS analyses, and the like also may be used in connection with the present disclosure. Other assays include immunoprecipitation of labeled ligands and immunocytochemistry, both in vitro and in vivo.

In certain embodiments, the immunological methods of the disclosure may detect the total VIPR2 protein or c-AMP level present in a subject and control samples.

In general, immunobinding methods include obtaining a sample suspected of containing a protein, peptide or antigen, and contacting the sample with an antibody or protein or peptide in accordance with the present disclosure, as the case may be, under conditions effective to allow the formation of immunocomplexes. Preferred samples, according to the present disclosure, include, but are not limited to, fluids, such as plasma, serum, cerebrospinal fluid, sputum, saliva, breast milk, tears, bile, semen, vaginal secretion, amniotic fluid, urine or stool sample, as well extracts of cells such as leukocytes, bone marrow cells, buccal cells, fibroblasts and tissue biopsies.

Contacting a biological sample with the protein, peptide or antibody under conditions effective and for a period of time sufficient to allow the formation of immune complexes (primary immune complexes) generally comprises adding the composition, for example an antibody, to the sample and incubating the mixture for a period of time long enough for the antibodies to form immune complexes with the VIPR2 protein or c-AMP. After this time, the VIPR2 protein- or c-AMP-antibody mixture will be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any radioactive, fluorescent, biological or enzymatic tags or labels of standard use in the art. U.S. patents concerning the use of such labels include U.S. Pat. Nos. 3,817,837; 3,850,752; 3,939,350; 3,996,345; 4,277,437; 4,275,149 and 4,366,241, each of which are incorporated herein by reference. A secondary binding ligand, such as a second antibody or a biotin/avidin ligand binding arrangement, as is known in the art, may also be used to detect the antibody-VIPR2 protein or antibody-c-AMP immunocomplex.

In certain embodiments, the primary immune complexes may be detected by means of a second binding ligand that has binding affinity for the VIPR2 protein or c-AMP or for the VIPR2 protein- or c-AMP-specific first antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody that has binding affinity for the VIPR2 protein or c-AMP is used to form secondary immune complexes, as described above. The second binding ligand contains an enzyme capable of processing a substrate to a detectable product and, hence, amplifying signal over time. After washing, the secondary immune complexes are contacted with substrate, permitting detection.

### ELISA

As a part of the practice of the present disclosure, the principles of an enzyme-linked immunosorbent assay (ELISA) may be used (Engvall and Perlmann, Immunochem., 1971; 8:871-873; Engvall, Methods Enzymol., 1980; 70 (A):419-39; Engvall, Lancet, 1976; 2(8000):1410; Engvall, Med. Biol., 1977; 55(4): 193-200; Gripenberg et al., Scand J. Immunol., 1978; 7(2):151-7). Such a method can be used for the detection and quantification of VIPR2 protein or c-AMP. ELISA allows for substances to be passively adsorbed to solid supports such as plastic to enable facile handling under laboratory conditions. For a comprehensive treatise on ELISA the skilled artisan is referred to “ELISA; Theory and Practice” (Crowther, Methods in Molecular Biology, 1995; 42:1-218, incorporated herein by reference). In general, in an ELISA, an antigen (or antibody specific for an antigen) is affixed to a surface, and then a specific antibody (or antigen) is washed over the surface so that it can bind to the antigen (or antibody). The antigen-antibody complex may then be detected, for example, by the conversion of a substrate to a detectable signal by an enzyme linked to the antibody, or through the use of labeled secondary or tertiary antibodies specific for the antigen-antibody complex.

The sensitivity of ELISA methods is dependent on the turnover of the enzyme used and the ease of detection of the product of the enzyme reaction. Enhancement of the sensitivity of these assay systems can be achieved by the use of fluorescent and radioactive substrates for the enzymes.

In certain embodiments, the disclosure comprises a “sandwich” ELISA, where anti-VIPR2 protein or ant-c-AMP antibodies are immobilized onto a selected surface, such as a well in a polystyrene microtiter plate or a dipstick. Then, a test composition suspected of containing VIPR2 protein or c-AMP, e.g., a clinical sample, is contacted with the surface. After binding and washing to remove non-specifically bound immunocomplexes, the bound antigen may be detected by a second antibody to the anti-VIPR2 protein or ant-c-AMP antibodies.

In certain embodiments, polypeptides from the sample are immobilized onto a surface and then contacted with the anti-VIPR2 protein or ant-c-AMP antibodies. After binding and washing to remove non-specifically bound immune complexes, the bound antibody is detected. Where the initial antibodies are linked to a detectable label, the primary immune complexes may be detected directly. Alternatively, the immune complexes may be detected using a second antibody that has binding affinity for the first antibody, with the second antibody being linked to a detectable label.

In certain embodiments, an ELISA in which the VIPR2 protein or c-AMP are immobilized utilizes antibody competition for detection. In this ELISA, labeled antibodies are added to the wells, allowed to bind to the VIPR2 protein or c-AMP, and detected by means of their label. The amount of VIPR2 protein or c-AMP in a sample is determined by mixing the sample with the labeled antibodies before or during incubation with coated wells. The presence of VIPR2 protein or c-AMP in the sample acts to reduce the amount of antibody available for binding to the well, and thus reduces the ultimate signal.

### 5.3 Methods of Treating Schizophrenia

In another embodiment of the present disclosure, a subject that has a VIPR2 CNV, an increased level of VIPR2 expression compared to a non-schizophrenic control, or an increased level of c-AMP compared to a non-schizophrenic control is a candidate for VIPR2 therapy, wherein an agent is administered in an amount effective to decrease the level of VIPR2 in a sample from the subject.

In certain embodiments the agent inhibits the function of VIPR2 protein or reduces the level of functional VIPR2 protein.

The agent can be administered, for example, systemically (e.g. by intravenous injection, oral administration, inhalation, etc.), by intra-arterial, intramuscular, intradermal, transdermal, subcutaneous, oral, intraperitoneal, intraventricular, or intrathecal administration, or may be administered by any other means known in the art.

In certain embodiments, the agent is a VIPR2 protein antagonist or inhibitor, for example, as described in Moreno, et al., 2000, Peptides 21:1543-1549; and Chu et al., 2009, Mol. Pharmacol. 77:95-101.

In certain embodiments, the VIPR2 protein antagonist or inhibitor is administered in an amount effective to reduce or inhibit the ability of VIPR2 protein to bind to VIP.

In certain embodiments, the VIPR2 protein antagonist or inhibitor is administered in an amount effective to reduce or inhibit the ability of VIPR2 protein to activate cyclic-AMP signaling, for example, cyclic-AMP accumulation, or protein kinase A (PKA) activation.

In certain embodiments, the VIPR2 protein antagonist or inhibitor is administered in an amount effective to reduce or inhibit the ability of VIPR2 protein to regulate synaptic transmission in the hippocampus.

In certain embodiments, the VIPR2 protein antagonist or inhibitor is administered in an amount effective to reduce or inhibit the ability of VIPR2 protein to promote proliferation of neural progenitor cells, for example, in the dentate gyrus.

In certain embodiments, the VIPR2 protein antagonist or inhibitor is administered in an amount effective to reduce or inhibit the ability of VIPR2 protein to modulate circadian oscillations in, for example, the suprachiasmatic nucleus.

In certain embodiments, the VIPR2 protein antagonist or inhibitor is an antibody that binds to the VIPR2 protein. An antibody can be a polyclonal or a monoclonal antibody composition. Such antibodies may also include but are not limited to chimeric, human, humanized, single chain, Fab fragments, and a Fab expression library. Means for preparing and characterizing antibodies are well known in the art and can be readily prepared through use of well-known techniques, such as those exemplified in U.S. Pat. No. 4,196,265, incorporated herein by reference (see also, e.g., Kohler and Milstein, Nature, 1975; 256:495-497; and Kohler and Milstein, Eur. J. Immunol., 1976; 6:511-519; Goding, Monoclonal Antibodies: Principles and Practice, 2d ed., Academic Press, Orlando, Fla., pp 60-61, 71-74, 1986; and Harlow and Lane, Antibodies, a Laboratory Manual, Cold Spring Harbor Laboratory, pp 139-281, 1988). Specifically, techniques developed for the production of chimeric and humanized antibodies have been described by Neuberger, et al., Nature, 1984; 312:604-608; Brüggemann et al., Proc Natl Acad Sci USA, 1989; 86(17): 6709-6713; and Takeda et al., Nature, 1985; 314:452-454. Various techniques have been described for the production of single chain antibodies including those in U.S. Pat. Nos. 5,476,786; 5,132,405; 4,946,778 each of which is incorporated herein by reference, and these techniques can be adapted to produce for example, VIPR2 protein-specific single chain antibodies. An additional embodiment of the disclosure may utilize the techniques described for the construction of Fab expression libraries of Huse et al., Science, 1989; 246:1275-1281, to allow rapid and easy identification of monoclonal Fab fragments with the desired specificity for a VIPR2 protein.

In certain embodiments, the agent is an antisense molecule, RNAi molecule or siRNA molecule. In certain embodiments, the antisense, RNAi or siRNA molecule is complementary to a segment or region of a VIPR2 mRNA transcript. In certain embodiments, the antisense, RNAi or siRNA molecule hybridizes to and inhibits or reduces translation of VIPR2 mRNA. In certain embodiments, the antisense, RNAi or siRNA molecule hybridizes to VIPR2 mRNA and increases degradation of the VIPR2 mRNA.

### 5.4 Kits

In further embodiments, the present application provides kits, such as an immunological kit, for use in detecting a VIPR2 CNV, VIPR2 nucleic acid or protein, and/or c-AMP in a biological sample. Such kits will generally comprise one or more oligonucleotides and/or antibodies that have specificity for VIPR2 CNV, VIPR2 nucleic acid or protein, and/or c-AMP.

In certain embodiments, a kit for detection of a VIPR2 CNV or VIPR2 nucleic acid will comprise, in suitable container means, one or more control VIPR2 CNV or control VIPR2 samples, and one or more oligonucleotide that specifically hybridizes to a DNA region associated with a VIPR2 CNV or VIPR2, as set forth above, for use in PCR, RT-PCR, qPCR, qRT-PCR, microarray analysis or nucleic acid sequencing. The kit may also comprise one or more polymerase, reverse transcriptase, and nucleotide bases, wherein the nucleotide bases may be further detectably labeled.

In certain embodiments, the oligonucleotide primers are immobilized on a solid surface or support, for example, on a nucleic acid microarray, wherein the position of each oligonucleotide primer bound to the solid surface or support is known and identifiable.

In certain embodiments, the immunodetection kits will comprise, in suitable container means, one or more control VIPR2 protein or control c-AMP sample, and one or more antibodies that bind to VIPR2 protein or c-AMP, and antibodies that bind to other antibodies via Fc portions.

In certain embodiments, the VIPR2 protein or c-AMP or primary anti-VIPR2 protein antibody or anti-c-AMP antibody may be provided bound to a solid support, such as a column matrix or well of a microtitre plate. Alternatively, the support may be provided as a separate element of the kit.

The immunodetection reagents of the kit may include detectable labels that are associated with, or linked to, the given antibody or antigen itself. Detectable labels that are associated with or attached to a secondary binding ligand are also contemplated. Such detectable labels include, for example, chemiluminescent or fluorescent molecules (e.g., rhodamine, fluorescein, green fluorescent protein, luciferase, Cy3, Cy5, or ROX), radiolabels (e.g., ³H, ³⁵S, ³²P, ¹⁴C, ¹³¹I)) or enzymes (e.g., alkaline phosphatase, horseradish peroxidase).

The kits may further comprise suitable standards of predetermined amounts, including both oligonucleotides, antibodies and VIPR2 protein or c-AMP. These may be used to prepare a standard curve for a detection assay.

The kits of the disclosure, regardless of type, will generally comprise one or more containers into which the biological agents are placed and, preferably, suitably aliquoted. The components of the kits can be packaged either in aqueous media or in lyophilized form.

The container means of the kits will generally include at least one vial, test tube, flask, bottle, or even syringe or other container means, into which the antibody or antigen may be placed, and preferably, suitably aliquoted. Where a second or third binding ligand or additional component is provided, the kit will also generally contain a second, third or other additional container into which this ligand or component may be placed.

The kits of the present disclosure will also typically include a means for containing the nucleic acids, VIPR2 CNV control samples, VIPR2 protein samples, c-AMP samples, or antibodies and any other reagent containers in close confinement for commercial sale. Such containers may include injection or blow-molded plastic containers into which the desired vials are retained.

### 6. EXAMPLES

### 6.1. Data Collection

To identify novel schizophrenia genes, copy number variation genome-wide was investigated using an approach that detects enrichment of multiple overlapping rare variants. An unbiased, genome-wide rare CNV analysis was performed using a staged approach. Regions of interest were defined in a primary sample of 802 patients and 742 controls, and statistical significance was estimated in a larger sample of 7,488 patients and 6,689 controls. The primary cohort consisted of unrelated patients derived from family-based studies conducted by investigators at the University of Washington, McLean Hospital, Columbia University, Trinity College Dublin, NYU, and Harvard Medical School (see Table 2); all samples were analyzed by array comparative genomic hybridization (CGH) using the NimbleGen HD2 platform. The secondary cohort consisted of Affymetrix SNP Array 6.0 data from the MGS study of schizophrenia³⁴, publicly available data from the International Schizophrenia Consortium (ISC), genotyped using Affymetrix 6.0 and 5.0 platforms²³, and Affymetrix 6.0 data on an independent set of controls from the Bipolar Genome Study (BiGS)³⁵, (see Table 2).

The Primary Cohort.

The initial discovery data set was composed of 1,761 unrelated subjects analyzed on the NimbleGen HD2 Array-CGH platform. The unfiltered sample consisted of 913 patients and 848 controls ascertained at ten sites (see Table 2). Ascertainment of these samples for family-based studies is described in previous publications¹⁻⁸. Microarray hybridizations using the NimbleGen HD2 platform were performed at the service laboratory of Roche NimbleGen according to the manufacturer's specifications. Samples were filtered from the dataset based on data quality measures described in Sections 6.4 and 6.5. Five duplicate samples from Trinity College Dublin also present in the ISC dataset were removed from the primary dataset. The final discovery data set consisted of 906 unrelated patients and 742 controls (see Table 2).

The Secondary Cohort.

The secondary cohort consisted of Affymetrix Genome-Wide Human SNP Array 6.0 data from the Genetic Association Information Network (GAIN) supported case-control studies “Genome-Wide Association Study of Schizophrenia” (phs000021.v2.p1) and the “Molecular Genetics of Schizophrenia—nonGAIN Sample” (phs000167.v1.p1). Both of these studies are subsets of the larger “Molecular Genetics of Schizophrenia Genome-wide Association” study, so the datasets were combined and the merged cohort is referred to as “MGS”. In total, the combined MGS sample consisted of 4,195 unrelated cases and 3,804 controls. A further 429 controls assayed on the Affymetrix 6.0 by the Bipolar Genome Study (BiGS) were joined with the MGS cohort to increase the number of controls. Genotyping of the MGS and BiGS cohorts was performed at the Broad Institute Center for Genotyping and Analysis (http://www.broad.mit.node/306).

Several NIMH controls overlapped between primary and secondary cohorts. Therefore, se eliminated overlapping samples from the dataset by retaining the experiment with the greatest probe density. Sample and experiment redundancy were minimized in the secondary cohort by prioritizing studies in order of GAIN-MGS>nonGAIN-MGS>BiGS. In total, 4,097 cases and 3,508 controls assayed on the Affymetrix 6.0 platform were included in stage two of the analysis. CNV data from the International Schizophrenia Consortium (ISC)⁸ was further combined with the processed data to increase the final size of the secondary cohort to 7,488 cases and 6,689 controls. Because sample identifiers were not available for CNVs in the ISC dataset, each CNV in the ISC call set was treated as coming from an independent sample. This approximation is reasonable given that the mean rare CNV burden in this dataset was less than one CNV per sample⁸.

### 6.2. Intensity Data Processing

With the exception of the published CNV calls from the ISC, all data were processed and analyzed centrally as follows. Microarray intensity data were normalized, and CNV calls were generated using an analysis package developed for the present study called C-score. All CNV call sets were filtered in a consistent fashion. In order to minimize the differential sensitivity of the various array platforms to detect CNVs, the analysis was limited to CNVs≧100 Kb. This size range is readily detectable by all platforms used in this study. The same criteria have been previously applied to filter CNVs across studies²³. Last, sensitivity to detect large (>100 Kb) copy number polymorphisms (CNPs) was evaluated at several locations in the genome, as described herein. Overall sensitivity to detect CNVs was comparable in cases and controls in both cohorts.

With the exception of data collected by ISC, all processing of microarray data was performed at Cold Spring Harbor Laboratory. Different methods were used for dual color intensity data from NimbleGen HD2 and single color intensity data from the Affymetrix Genome-Wide Human SNP 6.0 genotyping array.

NimbleGen HD2.

NimbleGen HD2 dual color intensity data were normalized in a two step process: (1) a spatial normalization of probes was performed to adjust for regional differences in intensities across the surface of the array, and (2) the Cy5 and Cy3 intensities were adjusted to a fitting curve by invariant set normalization.

Spatial normalization was performed using an R module provided by Roche-NimbleGen Inc. Invariant set normalization of intensity data involves selection of a probe set with minimal variability between the ranked test and reference autosomal probe intensities as described in Li et al¹⁰. The test intensities of this invariant autosomal probe set are then adjusted to the reference distribution. Based on these adjustments, a fitting curve is established to which all other intensities are shifted, preserving the variability in the data. The intensities of X and Y chromosomes were then extrapolated to the fitting curve. The process is repeated while exchanging the test and reference to simulate a dye swap experiment. The log 2 ratio for probe i is then estimated using the geometric mean of normalized and raw intensity data of test (Tst) and reference (Rfn) as follows:

$${log}_{2} ( {Ratio}_{i} ) = {log}_{2} ( \sqrt{\frac{{NormalizedTst}_{i}}{{RawRfn}_{i}} \times \frac{{RawTst}_{i}}{{NormalizedRfn}_{i}}} )$$

Affymetrix Genome-Wide Human SNP Array 6.0.

A two-step normalization method was developed to process Affymetrix SNP Array single color intensity data (Affymetrix 500K, Affymetrix 5.0 & Affymetrix 6.0). First, all arrays are normalized by invariant set normalization to a single reference array. To obtain the single reference array, the median-most array was selected based on autosomal intensities; then the correlation matrix was built for the adjacent 100 experiments (50 lower and 50 higher than the median-most). The single array most highly correlated with all others was selected as the common reference for Invariant Set Normalization based on the sum of Pearson's correlation coefficients. The reference array itself was not normalized. Second, for each experiment the ratio of intensities in comparison to a “virtual reference genome” (VRG) was calculated. The VRG for each “test” is a composite of multiple individuals from the study population and selected to minimize the overall variance in probe ratios. The VRG consisted of the median of the 10 most highly-correlated experiments in the dataset based on the Pearson correlation of 5% of probes on the array. This defined probe set was comprised of every 20th autosomal SNP probe and the nearest adjacent copy number (CN) probe. Separate virtual reference genomes were computed for males and females in order to have a sex-matched VRG.

$${log}_{2} ( Ratio ) = {log}_{2} ( \frac{NormalizedTst}{V R G} )$$

GC Correction of Log 2 Ratios.

The final step of data processing involved the correction of genomic waves effects in log 2 ratios due to regional correlations with GC content based on the fitted linear regression model proposed by Diskin et al¹¹.

### 6.3. Copy Number Discovery

In this study, a two segmentation algorithms was used to discover copy number variants in the GC-corrected NimbleGen HD2 and Affymetrix 6.0 ratio data, HMMSeg¹² and Genome Alteration Detection Analysis (GADA)¹³ (http://biron.usc.edu/˜piquereg/GADA). CNVs detected by both algorithms were used for downstream data processing and analysis. CNVs detected by only one algorithm were excluded. In addition, CNVs of the same type (i.e. deletion or duplication) that were separated by ≦3 probes were merged into one contiguous segment. The proximal and distal boundaries of overlapping and adjacent CNVs were defined by the minimal chromosomal start position and the maximal chromosomal end position of the CNVs. All CNV coordinates are based on the human genome build NCBI36/hg18.

### 6.4. Data Filtering

Data filtering criteria were designed to minimize the false discovery rates of rare CNVs in the primary and secondary analysis data sets. In this section the filtering of CNVs and experiments in the NimbleGen HD2 and Affymetrix data sets are described. Further details describing the filtering criteria of rare CNVs are outlined below in Section 6.6.

NimbleGen HD2.

Filtering of NimbleGen HD2 experiments from analysis was based on the experiment quality and filtered CNV properties. Experiments with autosomal median absolute deviations (aMAD)>0.23, with >400 filtered autosomal CNVs and with >30 Mb in combined CNV length (˜1% of the human genome) were removed. In addition, any sample with aneuploidy of autosomal chromosomes was excluded as were experiments with conflicting reported gender and empirically derived X and Y median probe ratios.

CNVs that overlapped regions of the genome prone to somatic cell rearrangements were removed from the primary data set. In particular, CNVs intersecting or overlapping T-cell receptor regions (chr7:38,245,705-38,365,141, chr7:141,647,285-142,221,100, chr9:33,608,462-33,652,656, chr14:21,159,896-22,090,937) and abParts (chr2:88,937,989-89,411,302, chr2:88,966,183-89,377,035, chr2:89,589,457-89,897,555, chr14:105,065,301-106,352,275, chr22:20,715,572-21,595,082) were excluded. CNVs with median probe ratios (seg.median) between 0.83 and 1.15 were also removed as were CNVs less than 100 kb or greater than 10 Mb.

Affymetrix 6.0.

Similar CNV and experiment filtering criteria used to process the primary NimbleGen HD2 dataset were implemented for analysis of CNVs in the secondary data set, taking into account the non-uniformity of Affymetrix 6.0 probe distribution across the human genome. Probable somatic T-cell receptor and abPart rearrangements were removed and CNVs were filtered out based on segment median thresholds. Noisy experiments with Median Absolute Deviation (MAD)>0.2 and chromosomal aneuploidies were eliminated from further analysis.

### 6.5. Rare CNV Formation

Within each cohort rare CNV frequencies were determined in the combined set of cases and controls (within each ethnic group separately). CNV frequencies were estimated based on 50% reciprocal overlap between CNV calls of the same type. CNVs with frequency >1% were removed.

Rare CNV calls which passed the 1% frequency filter were further filtered based on the confidence score (CS). Thresholds for CS were adjusted for various size classes of CNVs and for each platform separately as shown in Table 7. As the CS score of a CNV call, the P-value derived from the outlier detection genotyping method, median Z-score outlier detection (MeZOD)¹⁴ was used. Appropriate CS thresholds for each size class and platform were determined by examining patterns of Mendelian inheritance in a set of mother-father-child trios (of confirmed parentage), and CS was adjusted to achieve a 5% rate of Mendelian inconsistency for rare CNVs (i.e. 5% of CNVs called in the child were not inherited from a parent). This process maintained an error rate of 5% across a range of CNV sizes.

### 6.6. CNV Analysis

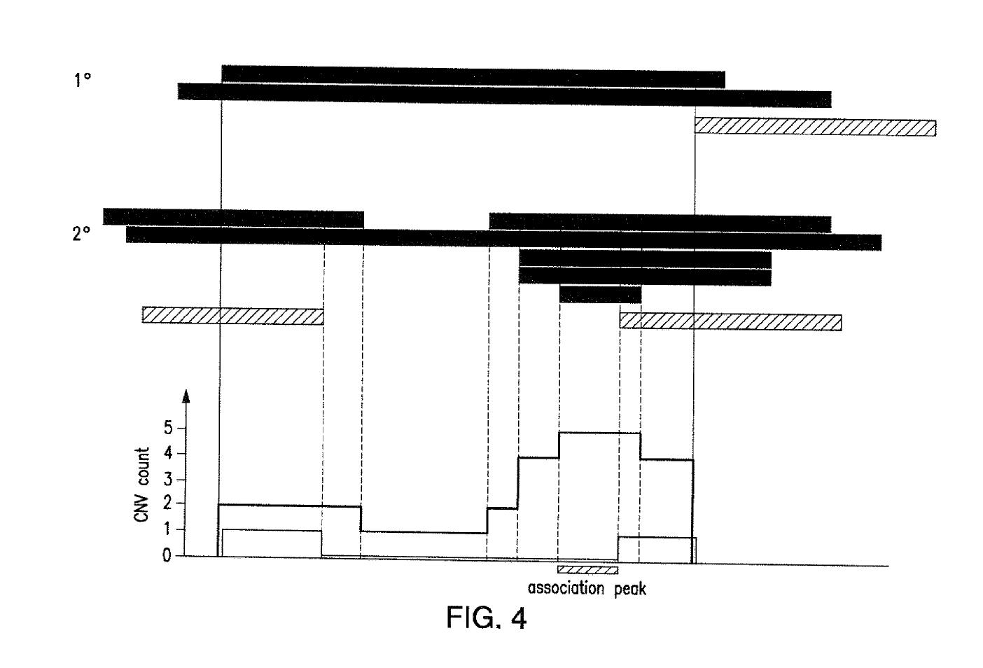

Regional CNV burdens associated with schizophrenia were analyzed using a two-stage approach. First, using the primary cohort, regions of interest were defined as all genomic segments containing CNVs in at least two cases and in no controls. This discovery step yielded 114 genomic regions of interest. In the secondary cohort of 7,488 patients and 6,689 controls, the association of these regions with schizophrenia were assessed (Table 3). All CNVs overlapping each of the 114 regions of interest were collected, and CNV breakpoints falling within each region were used to partition the region into a series of non-overlapping segments or bins (see FIG. 4). Significance was tested within each bin by exact conditional test, with ethnicity and study as covariates. The segment with the minimal p-value was defined as the peak of association within the region, and a permutation-based multiple testing correction scheme was applied in order to get the p-value for the region.

Based on the CNV counts within a segment, association was quantified using the Exact Conditional test, with ancestry and study as covariates. The segment with the lowest one-sided p-value was the peak of association within a ROI. Because segments in different ROIs are driven by the underlying genetic architecture, their numbers and sizes varied widely. Furthermore, numbers of CNVs in nearby segments are highly correlated. To address these issues, a permutation-based p-value correction scheme was applied, where the observed one-sided p-value of the association peak is compared to the distribution of minimal one-sided p-values of any segment within the ROI, computed based on data with case/control labels shuffled at the sample level. This empirical p-value is reported as the p-value for the ROI. In the experiments, p-value was estimated by running 100,000 permutations and p-values of the 8 nominally significant regions were further refined by running 200,000 permutations. For each ROI both the region and peak ORs estimated by the use of Exact Conditional test are reported. In order to avoid infinite OR estimates which occur when the number of controls across all strata is equal to zero, in those situations 0.5 was added to all cells in the contingency table (Haldane correction). In Table 1 and Table 3 Haldane-corrected ORs and the corresponding corrected peak p-values have been marked with an asterisk (*). Within the permutation-based procedure no correction of the counts has been performed, because a mixture of corrected and uncorrected minimal p-values which may occur within a ROI would interfere with the accurate estimation of the null distribution.

Of the 114 regions detected in the first step, 88 had events in the secondary cohort. Four regions had statistically significant associations in the secondary sample after Bonferroni correction (α=0.05/114=4.4×10−4). Table 1 lists the four regions with significant p-values under this criterion and an additional four loci with nominally significant p-values (4.4×10−4−0.05) in the secondary cohort. Regions with the significant associations were loss of copy number at 22q11.2 (P<5×10−6, OR=14.2), gain at 7q36.3 (P=4.0×10−5, OR=16.4), gain at 16p11.2 (P=1.0×10−4, OR=16.1) and loss at 15q13.3 (P=1.6×10−4, OR=14.9). No significant heterogeneity was observed for these genomic regions across the three studies (Table 1).

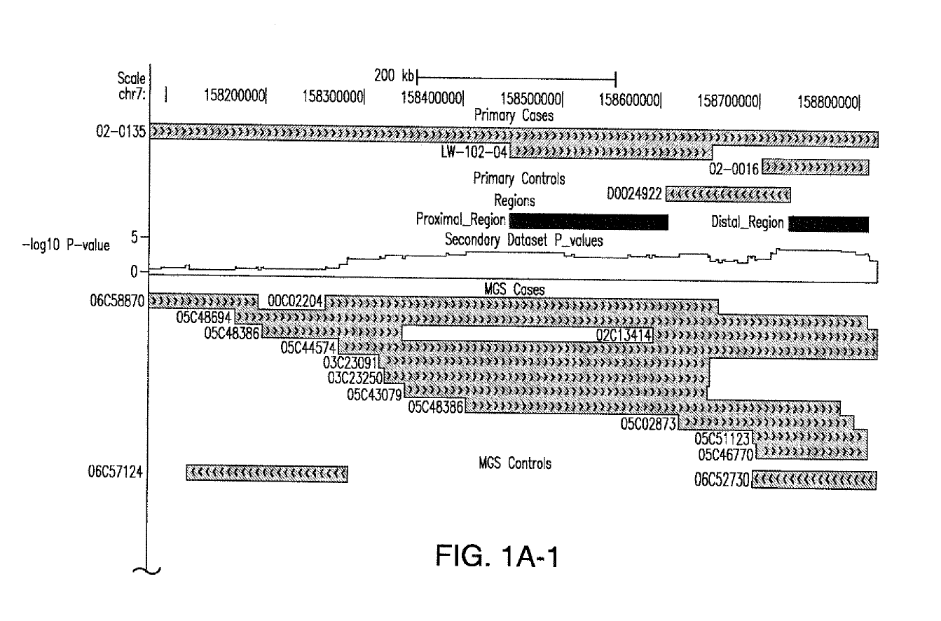

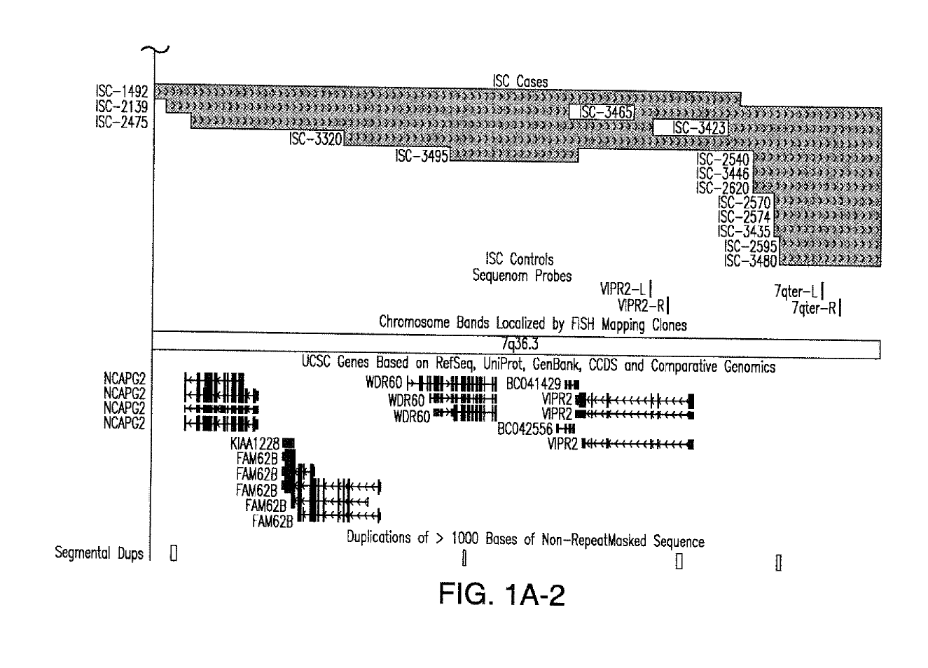

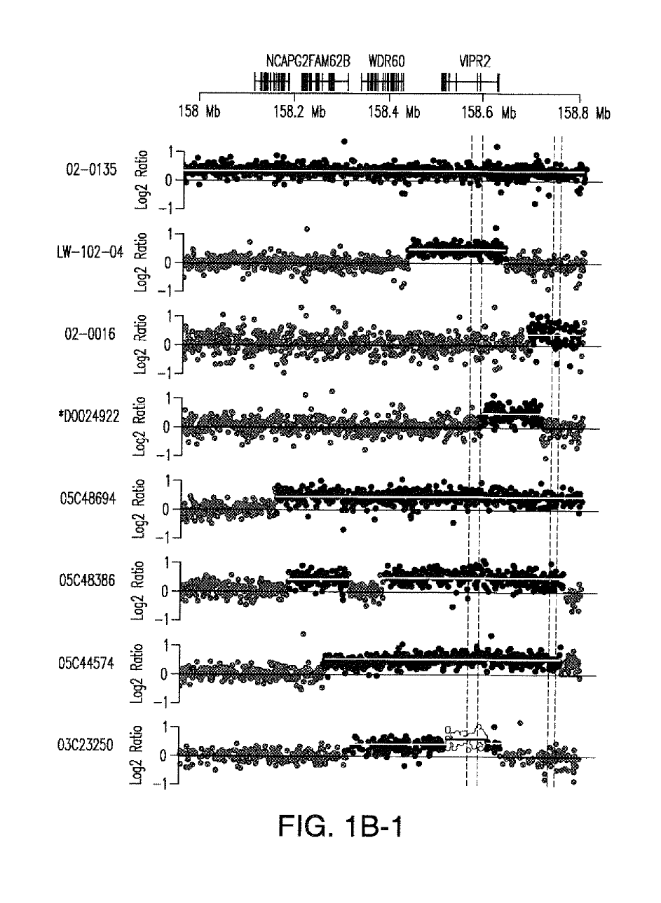

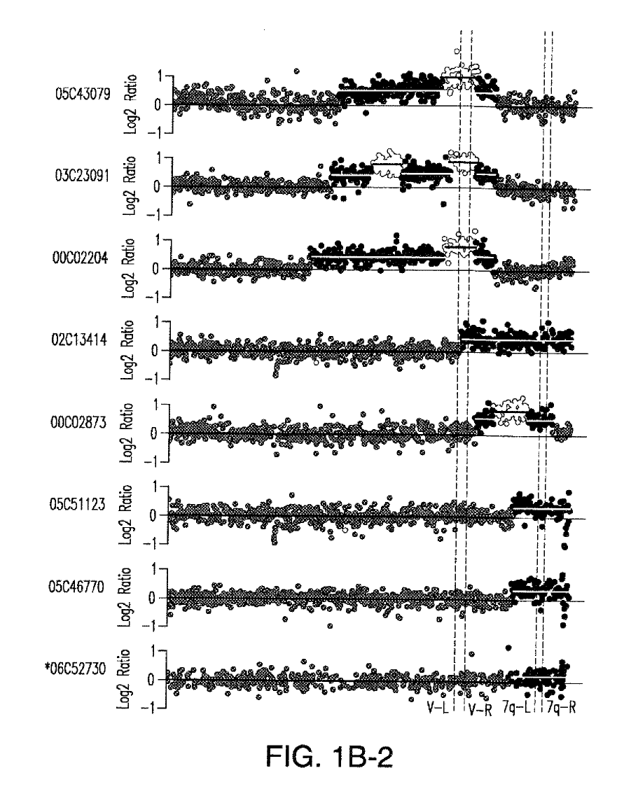

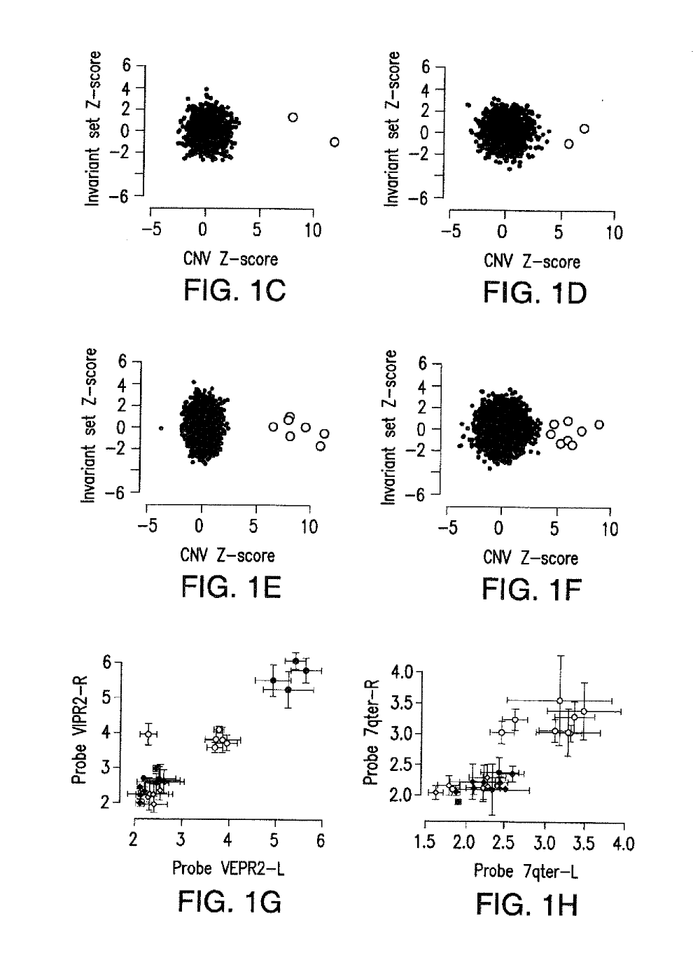

15q13.3, 16p11.2 and 22q11.2 are well-documented loci conferring increased risk for schizophrenia^{23, 24, 30}. All are hotspots for non-allelic homologous recombination (NAHR), and all alleles contributing to the association consist of large deletions with similar breakpoints. By contrast, microduplications at 7q36.3 have not been previously implicated in neuropsychiatric disorders. The 7q36.3 region harbored CNVs that overlapped but differed in size and breakpoint positions (FIG. 1a). The peak of association is located in the subtelomeric region of 7q, upstream of the gene VIPR2. Also, ranking fifth among the associations genome-wide was another region, 125 kb proximal to the peak at 7q36.3 (P=0.0007, Table 1). Ten individuals carried duplications of this region that did not overlap with the peak of association. Combining the two 7q36.3 regions into a single 362 Kb region (chr7:158,448,321-158,810,016), duplications were detected in 29 of 8,290 (0.35%) patients and 2 of 7,431 (0.03%) of controls in this study. The p-value for the combined region in the combined sample was 5.7×10−7 and the odds ratio was 14.1 [3.5, 123.9]. A complete list of 7q36.3 CNVs is provided in Table 4.

### 6.7. Experimental Validation 7q36 CNV

Sequenom MassArray. Absolute copy number (ACN) detection method by Sequenom MassArray combines real time competitive PCR (rPCR) with MassEXTEND procedures and matrix-assisted laser desorption/ionization time-of-flight mass spectrometry (MALDI-TOF) (http://www.sequenom.com). The CNV assay involves spiking genomic DNA template with genomic DNA from a single reference chimpanzee (Pan troglodytes) that was purchased from Southwest National Primate Research Center, (San Antonio, Tex., USA). The genotype assay targets a specific single nucleotide difference between human and chimpanzee, and relative copy number is determined based the ratio of peak areas of the human and chimp alleles.

CNV Assay Design: CNV assays were designed for two segments (proximal and distal) of the 7q36.3 region as shown in FIG. 1. Two assays were designed for each segment. These assays were used to validate all CNV calls, with the exception of one duplication in control sample D0024922 that did not overlap with either segment. For this CNV region a 3rd pair of assays was developed. In addition, similar assays were developed for 4 copy number invariant regions of the genome. Assays were designed to be carried out in a single multiplex reaction.

Sequence differences between human and chimpanzee were identified within CNV target regions by aligning the respective genomic DNA using UCSC Blat (http://genome.ucsc.edu). Nucleotide differences between human and chimpanzee sequences were then categorized based on the position of difference in the alignment, alleles in human and chimpanzee, and direction of alignment. All aligned human-chimpanzee loci were processed to identify the location of any variant bases (single nucleotide polymorphisms and insertions/deletions) within a given distance to the specified human-chimpanzee loci. All known human and chimpanzee SNPs and indels were excluded in CNV assay design. Single base nucleotide extension (SBE) assays were then designed to target the non-conserved nucleotides using Sequenom Assay Design v3.1 software. The primers used for Sequenom assays are listed in Table 7.

PCR: PCR was performed on Biorad thermocyclers in a 5 μl reaction using 15 ng of genomic human DNA (hsDNA), 15 ng of P. troglodytes DNA (ptDNA), 0.5 pmol of each primer in multiplex, 250 μM dNTPs, 0.5 μl of 10×20 mM MgCl PCR Buffer and 0.5 units of Taq polymerase (Roche, USA). The PCR cycling parameters were 95° C. for 15 min, followed by 45 cycles of 94° C. for 30 s, 56° C. for 30 s and 72° C. for 1 min, followed by 72° C. for 3 min. Following PCR, 5 μl PCR product was treated with 0.17 μl of 10×SAP (shrimp alkaline phosphate) buffer and 0.30 μl of SAP enzyme and incubated at 37° C. for 40 min followed by heat denaturation of enzyme at 85° C. for 5 min.

Single Base Primer Extension: Primer extension was performed on MJ thermocyclers in 9 μl reactions using the 7 μl SAP-treated PCR product, 7-14 pmol of each primer in multiplex, 0.20 μl of iplex buffer, 0.20 μl of iplex termination mix and 0.05 μl of iplex enzyme (Sequenom, San Diego, Calif., USA). The cycling parameters were 94° C. for 30 s, followed by 40 cycles of 94° C. for 5 s and a nested 5 cycles of 52° C. for 5 s and 80° C. for 5 s, followed by 72° C. for 3 min.

Quantification of absolute copy number: The absolute copy number for target regions was determined using the method described by Williams et al¹⁵. All copy number measurements from each sample were normalized against copy number measurements from the assays targeting the invariant (normal diploid copy) regions of the genome in the same sample to control for any sample to sample (i.e. well to well) loading variation. Data were analyzed using Splus 8.0.

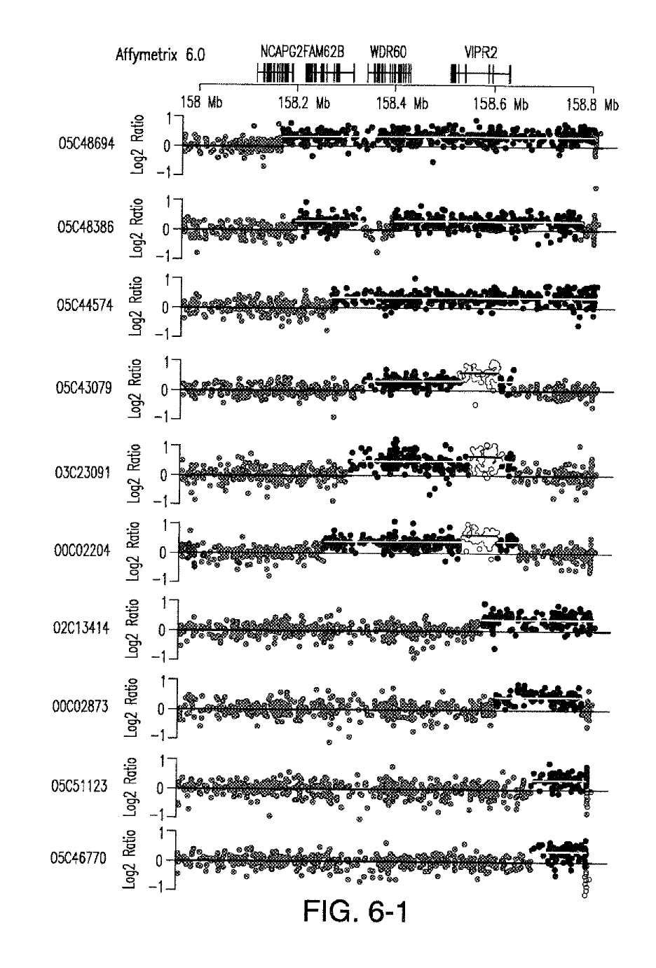

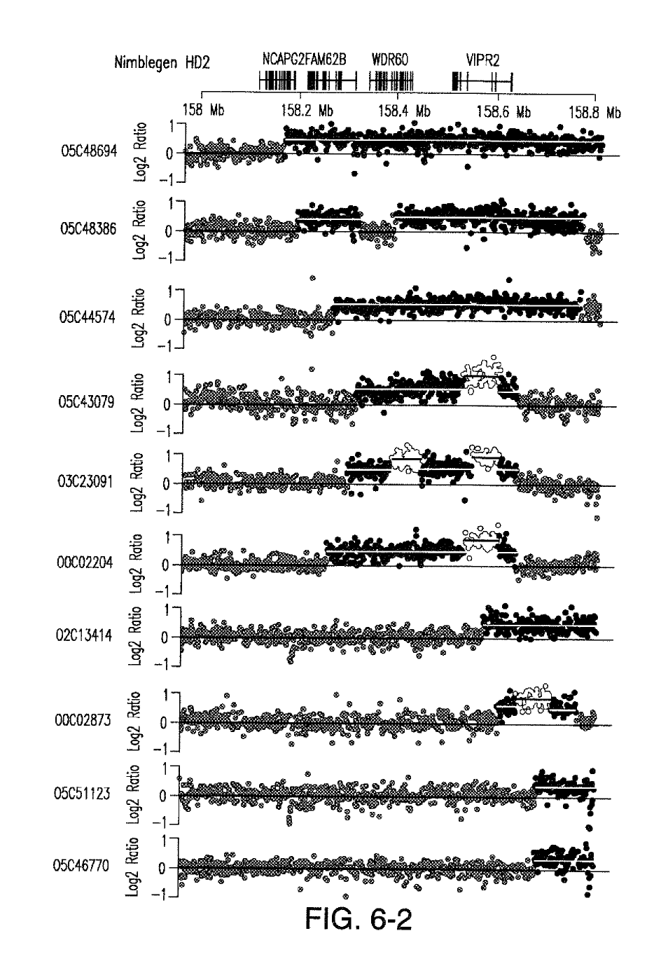

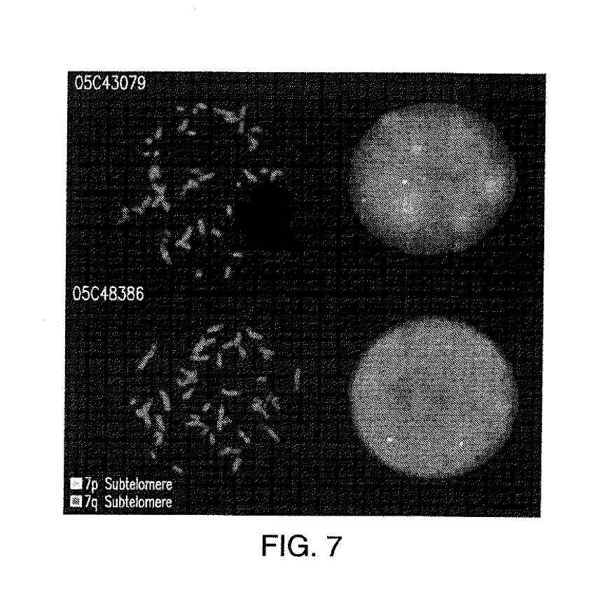

As outlined in detail below, sensitivity and specificity of CNV calls in the 7q36 region were examined to determine the possibility of a spurious association. No additional duplications >100 kb were detected after reducing the stringency of the CNV filtering criteria. Second, a more sensitive targeted CNV calling algorithm, median Z-score Outlier Detection (MeZOD)³⁰, was applied to obtain CNV genotype calls for the proximal and distal regions of 7q36.3. All 16 copy number gains, nine overlapping the proximal region (FIG. 1c,e) and ten overlapping the distal region (FIG. 1d,f) formed a punctuate cluster distinguishable from the overall distribution of Z-scores, but no additional duplications were identified. Based on these results, it is unlikely that the segmentation-based CNV calling algorithms failed to detect duplications >100 Kb in cases or in controls. Specificity of CNV calls was confirmed by experimental validation. All 16 duplications detected in the primary and secondary cohorts were tested using the Sequenom MASSarray genotyping platform (detailed below). All but one of the duplications (control sample 06C52730) were confirmed using the Sequenom MASSarray genotyping platform with assays designed for the proximal region (FIG. 1g) and for the distal region (FIG. 1h), and control sample D0024922 was validated using a third set of probes. CNVs that were validated in the MGS subjects were mapped at higher resolution using the NimbleGen HD2 platform (as outlined below), and plots of probe intensity ratios from the HD2 array are shown in FIG. 1b and FIG. 6. In addition, tandem duplications of the VIPR2 gene were confirmed in two patients by fluorescence in situ hybridization (FISH) (FIG. 7).

Unexpectedly, manual examination of probe ratios in FIG. 1b revealed additional structural complexity within some of the 7q36.3 CNVs. Copy number profiles in four patients (03C23250, 05C43079, 03C23091 and 00C2204) suggested triplications nested within duplications of the proximal region (FIG. 1b). In all four patients, the nested triplication overlapped with exons 3 and 4 of the gene VIPR2. A copy numbers of four was confirmed in these samples using the Sequenome MASSarray CNV assay (FIG. 1g), and results for all samples were consistent with results in FIG. 1b. VIPR2 transcripts were amplified from mRNA samples from the four triplication carriers. The normal VIPR2 transcript was detected in all samples, and we did not observe a larger product corresponding transcript with duplicated exons.

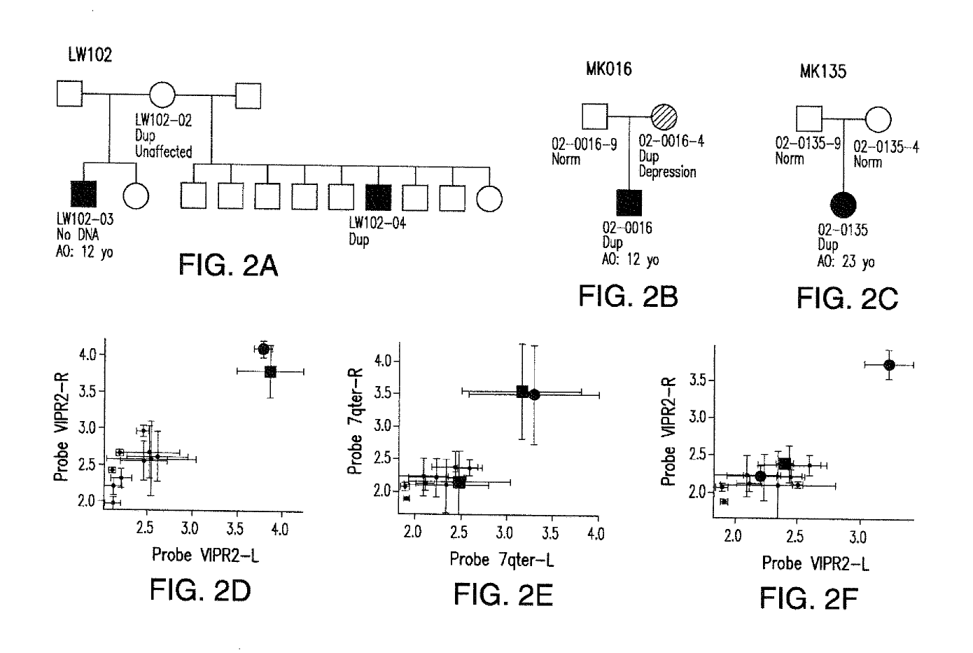

Inheritance of the duplication at 7q36.3 could be evaluated in three families (FIG. 2). In family 02-135, the duplication was confirmed in the proband, but not detected in either of the unaffected parents, and thus is apparently de novo (FIG. 2f). In family 02-016 duplication was detected in the proband and in a mother with a diagnosis of depression (FIG. 2d). In family LW102, duplication was detected in the proband and in an unaffected mother; this mother also had son with a diagnosis of schizophrenia (LW-102-03) from a second marriage, but DNA was not available on this individual. Clinical psychiatric reports of patients 02-016 and 02-135 are provided in section 6.12, below.

Fine Mapping of 7q36.3 Duplications in MGS Subjects Using the Nimblegen HD2 Array.

Duplications of 7q36.3 detected by the Affymetrix 6.0 array in the MGS sample were mapped at finer resolution using the Nimblegen HD2 platform. (FIG. 6). Labeling and Hybridizations were performed as described in section 1 above.

Based on these results (FIG. 6) most duplications have unique boundaries, suggesting that each is a result of a different mutation event. Furthermore, the Nimblegen data reveal additional structural complexity, including additional triplicated regions in samples 03C23091 and 00C2873 that were not readily apparent from the original Affymetrix scans.

Fluorescence In Situ Hybridization (FISH).

Cytogenetic confirmation was obtained for 2 samples with duplication of VIPR2 (05C43079 and 05C48386). Subtelomeric probes were obtained for chromosome 7p and 7q (Abbott Molecular). The genomic coordinates of the TelVysion 7q probe were Chr7:158551912-158650925 (hg18), which overlaps exons 1-4 of VIPR2. Hybridizations were performed according to manufacturer's protocols.

In images from fluorescence in situ hybridization (FISH) using a subtelomeric 7q probe, three distinct red signals can be seen in interphase cells, confirming a heterozygous duplication. In metaphase cells, all signals localize to the subtelomeric region of 7q, confirming that the duplications lie adjacent to each other in the 7q36.3 region (FIG. 7).

### 6.8. Critical Evaluation of CNV Detection Sensitivity in Cases and Control Across the Primary and Secondary Cohorts

Sensitivity of CNV calls in the 7q36.3 region were examined to determine the possibility of a spurious association. A spurious association can arise in CNV data, for instance, if there is comparatively reduced sensitivity to detect CNVs in the control sample compared with the case sample. Sensitivity of the segmentation-based CNV calling methods was evaluated by first examining CNVs within the 7q36.3 region after relaxing the stringent filtering criteria, and then by comparing segmentation calls in cases and controls to CNV genotype calls made in the 7q36.3 region and elsewhere in the genome using targeted genotyping algorithms with enhanced sensitivity. These efforts were focused only on the microarray datasets that were processed for this study. Published CNV call sets generated by the ISC study of schizophrenia have undergone evaluation in earlier studies⁸.

CNV Segmentation Calls in the 7q36.3 Region Examined Using a Lower Sensitivity Threshold.

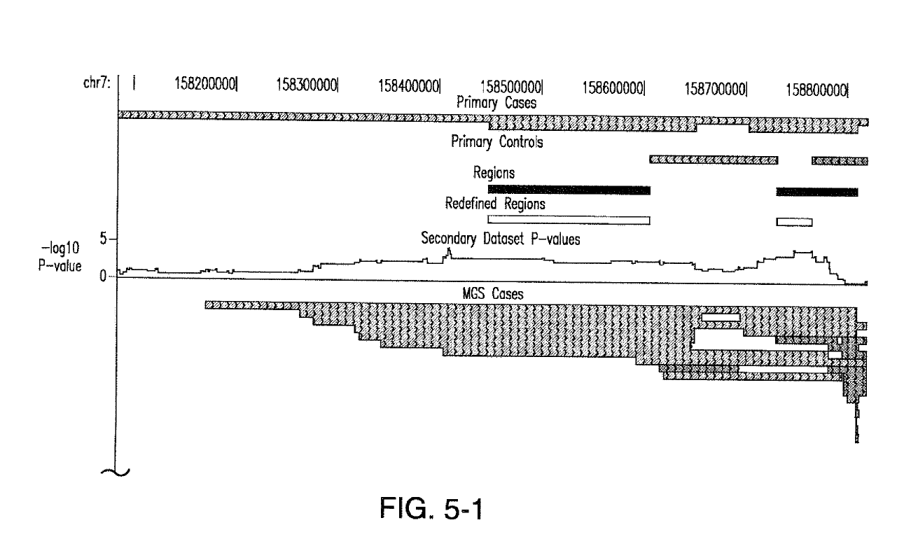

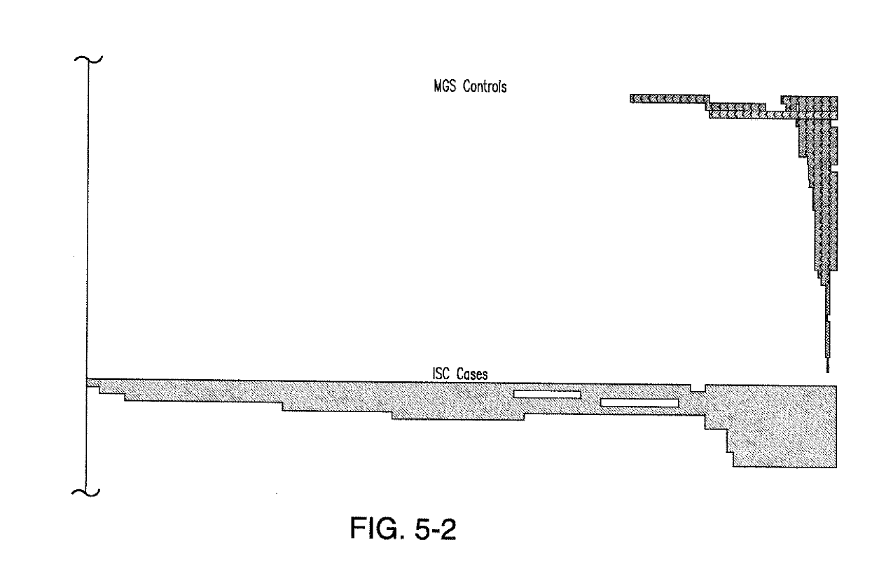

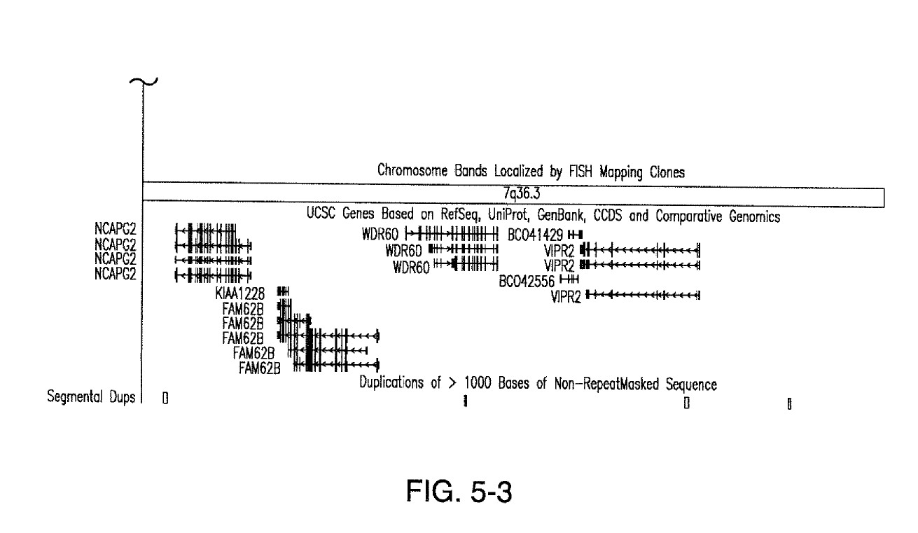

After eliminating the primary filtering criterion, the CNV confidence score (CS), no additional CNVs were detected in the 7q36.3 region; therefore, filtering based on confidence score does not account for the observed differences in CNV frequencies. Second, rare CNVs in the 7q36.3 region were examined after relaxing the minimum size to 5 probes and 16 probes in the Nimblegen and Affymetrix data respectively. Additional small CNVs were detected that did not negatively influence the observed association (FIG. 5). The two-stage analysis of the 7q36.3 region was repeated using the unfiltered CNV call set, and the statistical signal was slightly improved compared with the original result from the stringently filtered CNV calls (permutation P-value=1.5*10−5, Peak OR=17.1 [3.3, Infinity]). This improved signal can be accounted for by the smaller target region and reduced multiple testing correction. The improved signal cannot be accounted for by the detection of additional small CNVs in cases, because only 3 smaller duplications overlapped the peak of association, one in cases and two in controls.

Concordance Between Segmentation Calls at 706.3 Region and CNV Calls by a More Sensitive Outlier-Detection Based CNV Detection Method (MeZOD).

As a second approach to evaluating the sensitivity of the disclosed methods, a more sensitive outlier-detection based method, Median Z-score Outlier Detection (MeZOD), was applied to the analysis of the two CNV target regions at 7q36.3. Outlier detection-based methods^{14, 16} provide greater sensitivity for CNVs within defined target regions. If a significant number of CNVs overlapping the target region were undetected by the segmentation algorithms or filtered out of the call set, such variants can often be detected by MeZOD, leading to discordance between the segmentation calls and the MeZOD genotypes. When the distribution of z-scores for each target region was examined, punctuate clusters of data points were observed that are distinguishable from the overall distribution (displayed as cluster plots in FIG. 1). Perfect concordance between MeZOD genotypes and CNV calls was observed. Based on these results, it is unlikely that the segmentation-based CNV calling algorithms failed to detect CNVs within this region that are >100 kb in size.

Sensitivity to Detect CNPs is Comparable in Cases and Controls.

Sensitivity at other loci throughout the genome was evaluated by examining sensitivity to detect a set of validated common CNPs (>100 Kb in size) characterized as part of HapMap phase 3¹⁷. Sensitivity to detect common CNPs by the segmentation algorithms is equivalent to that of rare CNVs (because parameters of HMM-based segmentation algorithms are not adjusted based on prior knowledge of common CNVs). However, targeted genotyping methods have much greater sensitivity and accuracy. Therefore concordance between the segmentation calls and a predefined set of common CNP genotypes was used as a measure of the segmentor sensitivity,

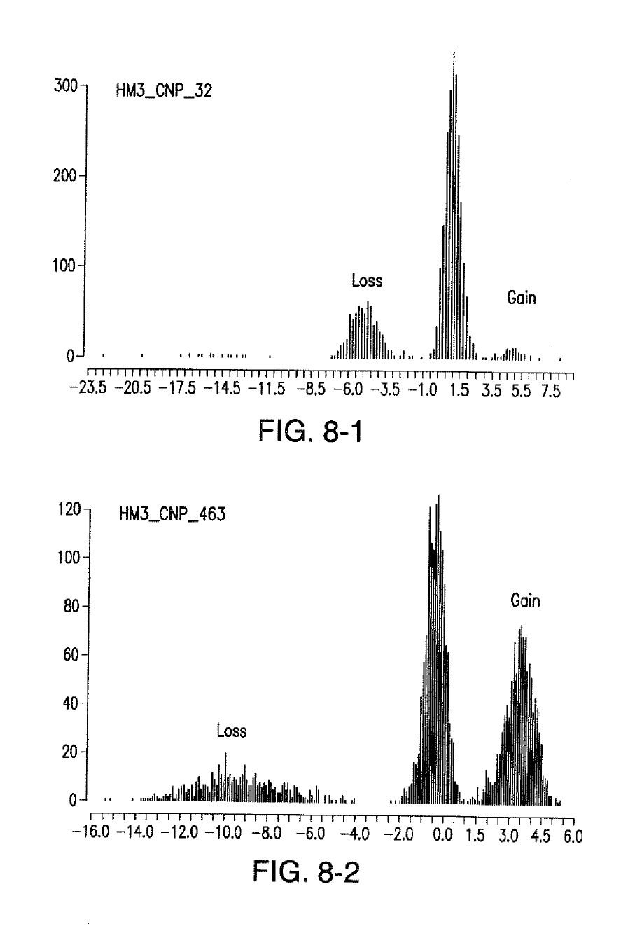

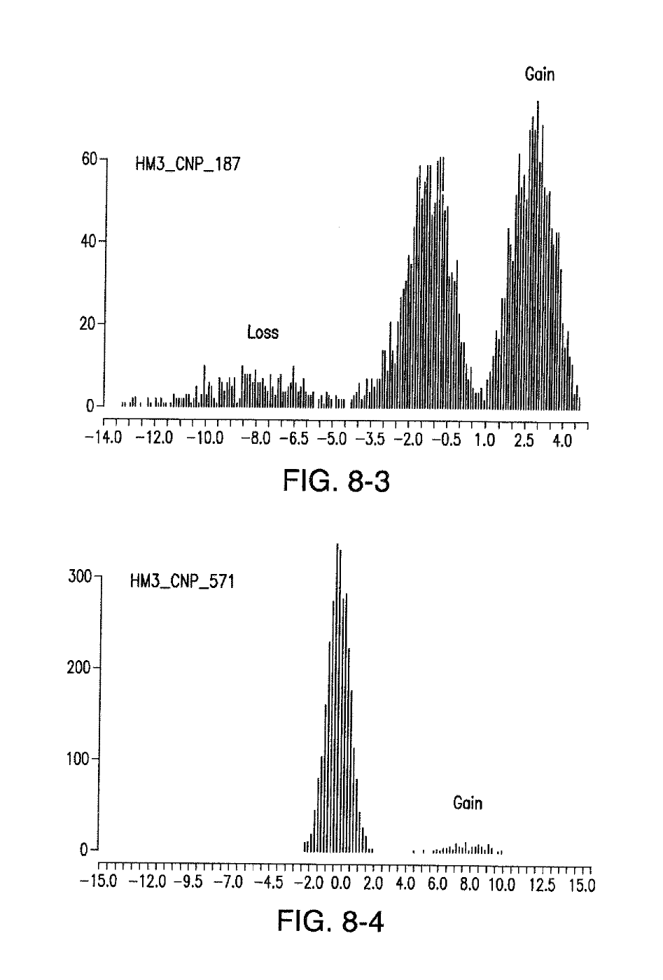

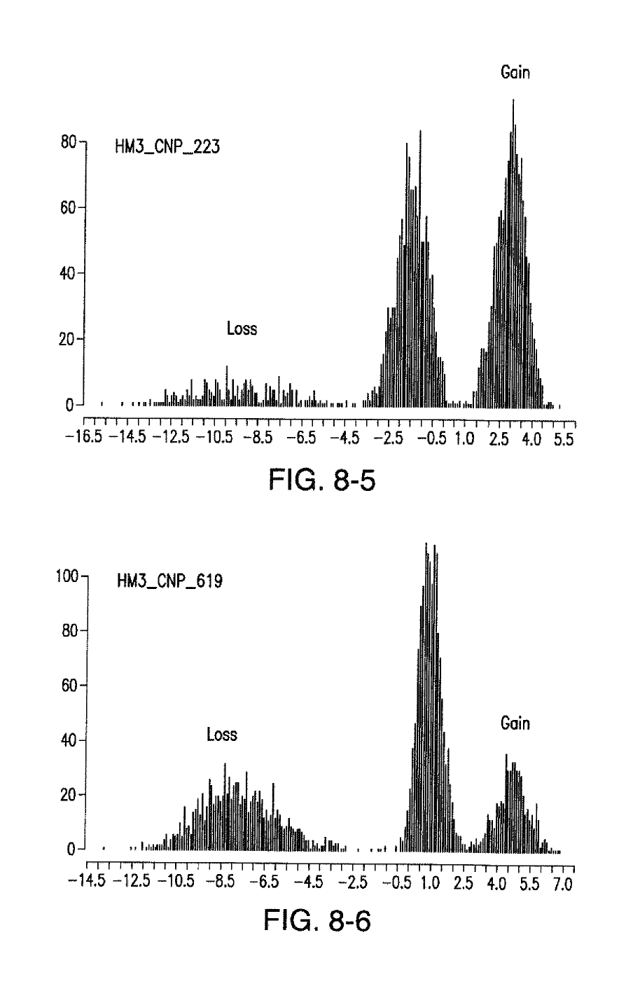

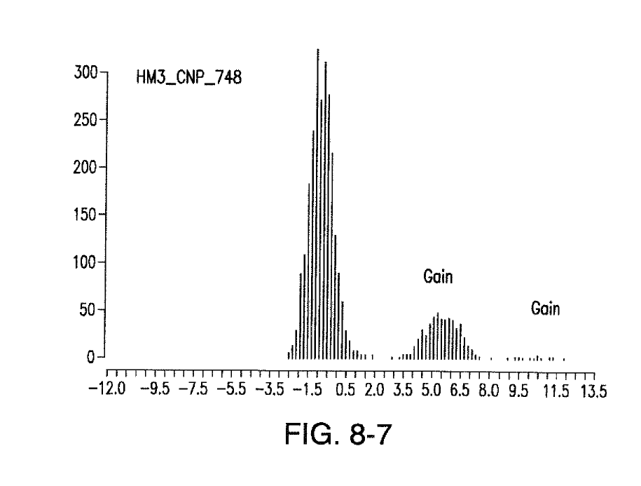

First, all common CNPs >100 Kb were selected from HapMap 3 (http://hapmap.ncbi.nlm.nih.gov/downloads/cnv_data/hm3_cnv_submission.txt), and these markers were compared with a list of common CNP markers that were previously assembled for the NimbleGen HD2 platform. All CNPs that were present in both sets (total of 7) were selected. The seven CNPs were then genotyped by manual examination of clusters as illustrated in FIG. 8. Genotypes were compared with segmentation calls, and sensitivity was defined as the average fraction of genotyped “gains” or “losses” that were detected by segmentation per individual. By this approach, similar overall sensitivity were observed between cases and controls in the primary sample and in the MGS samples (Table 7), with the possible exception that sensitivity was slightly (but significantly) greater in African American controls compared with cases in the MGS study (Case:Control ratio 0.96, P=0.001)

Examining the Impact of Differential Sensitivity of Platforms Used in Different Studies.

The primary dataset consisted of NimbleGen 2.1M array data. The secondary dataset consisted of the MGS study (Affymetrix 6.0) and the ISC study of schizophrenia (which included data from Affymetrix 5.0 and 6.0 platforms). Combining data from multiple studies has the potential to create spurious associations. For instance, if data from two studies are combined and the study with the most sensitive platform had a large number of cases and a small number controls, a spurious enrichment of CNVs in cases could arise (if factors such as platform and study are not controlled for). There was no such a disparity in any of the 3 studies comprising the primary and secondary cohorts, which consisted of roughly equal numbers of cases and controls (Table 2). In the conditional Fisher's exact-based permutation test for association, platform and study were included as factors. Second, heterogeneity in the association across all 3 studies was tested for in the primary and secondary samples; no significant heterogeneity was observed across the 3 populations when tested using the Breslow-Day Tarone test (P=0.19−0.86). Therefore, evidence that the observed association can be attributed to differential sensitivity of platforms used across studies was not found.

### 6.9. Quantitative PCR Measurement of VIPR2 mRNA

Levels of VIPR2 mRNA were measured in lymphoblastoid cell lines obtained from the MGS study collection at the NIMH genetics repository (http://www.nimhgenetics.org), including patients with duplications and triplications of 7q36.3 and control individuals with normal diploid copy number of this region. The samples and genotypes used in these experiments were as follows: controls: 04C34751, 04C28567, 05C47472, 04C34070; duplication of VIPR2: 05C44574, 05C48386, 05C48694; Duplication of VIPR2 with a nested triplication of exons 3 and 4: 00C2204, 03C23250, 05C43079, 03C23091; duplication of upstream subtelomeric region: 05C51123, 05C46770. CNV genotypes were confirmed in cell lines by Sequenom assay (see Section 6.8 and FIG. 1).

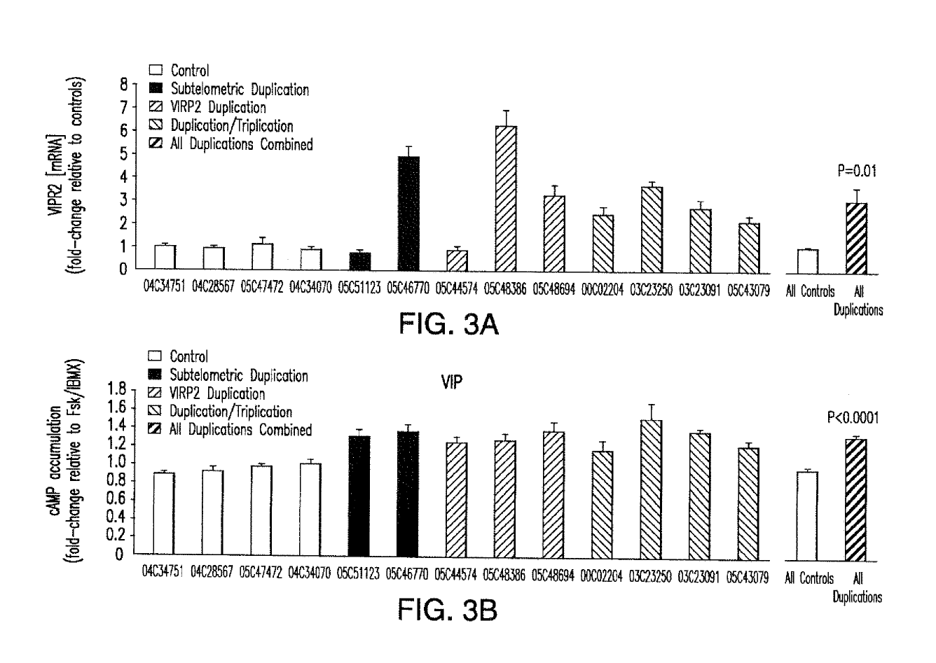

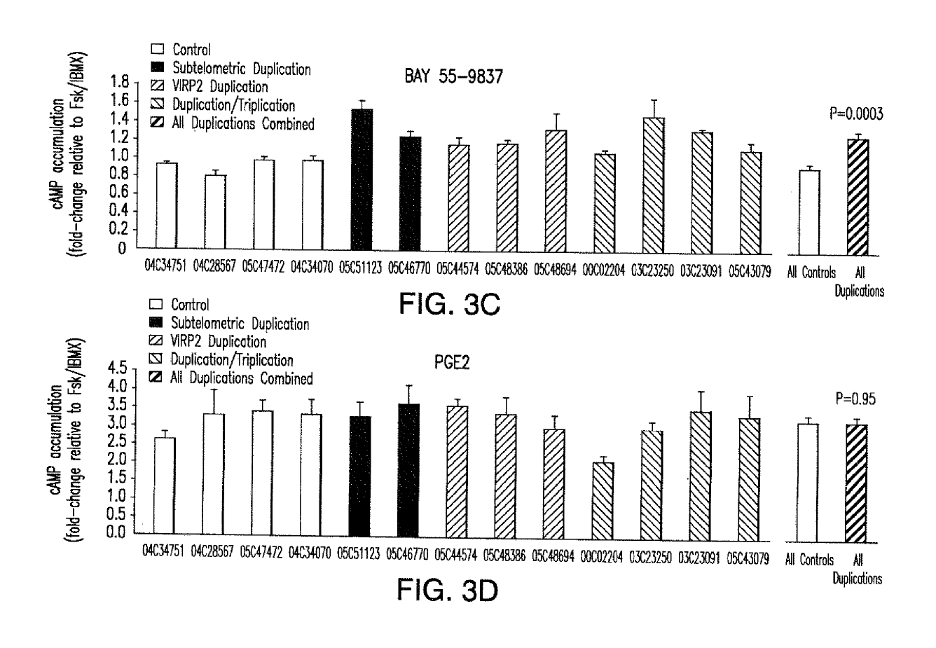

Cell lines from two subjects with each genotype were analyzed. From each cell line, a series of two RNA preparations were made in order to provide two biological replicates of each subject. Each biological replicate was in turn analyzed with triplicate qPCR reactions. Results presented for each subject represent the mean and standard error of 6 replicates. Standard error and P-values for pooled results were computed across individuals (FIG. 3)

Tissue Culture.

EBV-transformed lymphoblastoid cells from MGS patients and controls were obtained from the NIMH genetics initiative repository. Cell lines were cultured in Gibco's RPMI-1640 supplemented with 15% FBS and 1× Penn-Strep at 37° C. and 5% CO2.

Sample Preparation.

Total RNA was prepared using Qiagen's RNEasy Plus kit, and cDNA was prepared using Quanta Qscript cDNA mix using 1 ug RNA for each 20 μl reaction.

Quantitative PCR (qPCR).

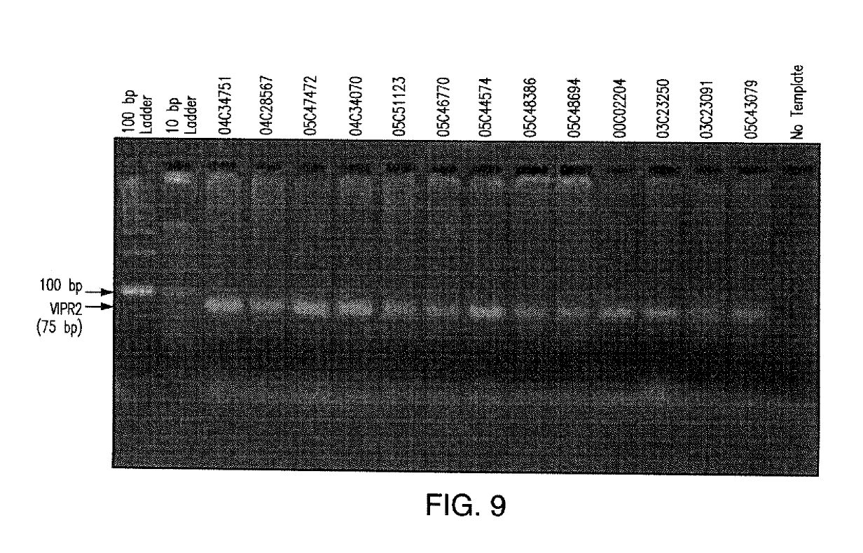

Measurement of VIPR2 mRNA abundance was carried out on mRNA from lymphoblastoid lines using the VIPR2 TaqMan® assay (Applied Biosystems Inc.). Assays were carried out by the Center for Aids Research (CFAR) core facility at UCSD (http://cfar.ucsd.edu/core-facilityes/genomics-core/protocols-and-resources-1/quantitative-real-time-rt-per-sample-preparation). cDNA from 500 ng of total RNA starting material was used for each qPCR reaction. Samples were done in triplicates using the VIPR2 and GAPDH primers provided by the manufacturer. qPCR cycling conditions were as follows: 50° C. for 2 min, 95° C. for 10 min, then 45 cycles of 95° C. for 15 sec, 60° C. for 1 min. Ct values obtained were normalized with GAPDH Ct values. VIPR2 mRNA was detectable in all cell lines analyzed in this study (FIG. 9).

Low but measurable levels of VIPR2 expression was observed in all lymphoblastoid cell lines in this study (the range of Ct values was 33-36). This is consistent with the low VIPR2 transcript abundance in the published transcriptome data from the cell lines of 60 Nigerian subjects by Pickrell et al (21 reads/968 million) 18. This is also consistent with the very low level of VPAC2 receptor signaling observed in control cell lines.

In cell lines from patients carrying duplications of terminal 7q, an approximately 3-fold increase in VIPR2 expression relative to controls was observed (FIG. 3A).

### 6.10. Measurement of VIP-Induced cAMP Signaling in Lymphoblastoid Cell Lines

Cyclic-AMP signaling has been implicated in schizophrenia^{51, 52}. It was determined whether increases in VIPR2 transcription and VPAC2-mediated cAMP signaling was a consequence of the microduplications at 7q36.3. VIPR2 mRNA and cAMP accumulation in response to VIP and a VPAC2-selective agonist (BAY 55-9837) in lymphoblastoid cell lines from eight MGS study subjects was assessed: two with subtelomeric duplications, three with duplications of VIPR2, four with partial triplications, and four controls with normal copy number of the region.

Cyclic AMP accumulation was measured in lymphoblastoid cell lines (0.5 million per ml) pre-incubated for 20 minutes with the cyclic nucleotide phosphodiesterase inhibitor isobutylmethylxanthine (IBMX, 200 μM), before the addition of the stimulatory agonists forskolin (10 μM)+/−VIP, [100 nM], BAY 55-9837 (100 nM) or prostaglandin E2 [PGE2, 1 μM] for 10 min. Reactions were terminated by pelleting the cells, aspiration of the medium and addition of 100 μl of cold 7.5% (wt/vol) trichloroacetic acid (TCA). Cyclic AMP content in TCA extracts was determined by radioimmunoassay and normalized to the number of cells per well. Data are expressed as cAMP accumulation in response to the GPCR agonists relative to the response to non-GPCR agonist forskolin (10 μM) and IBMX (200 mM) alone. Results presented for each subject represents the mean and standard error of at least ten replicates. Standard error and P-values for pooled results were computed across individuals (FIG. 3).

VIPR2 transcripts were present at detectable levels in all cell lines. VIPR2 mRNA levels were significantly increased in duplication carriers compared to controls (FIG. 3a). Likewise, cAMP responses to VIP and the BAY 55-9837 were significantly greater in lymphoblastoid lines from carriers as compared to controls (FIG. 3b). In contrast, no group difference in cAMP accumulation in response to a different GPCR agonist, prostaglandin E2, was observed, thus confirming that the effect of 7q36 duplications on cAMP accumulation is mediated by VPAC2R.

In cell lines from patients carrying duplications of terminal 7q, a significant increase in cyclic-AMP accumulation in response to two different VPAC2 agonists relative to controls was observed (FIGS. 3B and 3C). No difference in cyclic-AMP response was observed in response to a different GPCR agonist PGE2 (FIG. 3D).

### 6.11. Sequencing of Transcripts in Patients with Duplications of VIPR2 Exons 3 and 4

In four MGS patients, complex duplication/triplication were observed consisting of a duplication of the entire gene and another duplication of exons 3 and 4 nested within it. The exact structure of this haplotype is not known. If the nested duplication of exons 3 and 4 lies adjacent and in a direct orientation, then this rearrangement may result in a mutant transcript with tandem duplication of exons 3 and 4 which would result in a shift of the open reading frame. However, if the nested duplication lies outside of the gene or in an inverted orientation, then nested duplication would not have an obvious impact on either of the two copies of VIPR2 (thus, the duplication/triplication might be functionally equivalent to a tandem duplication).

Using primers designed for exons 2 and 5, VIPR2 cDNA products were sequenced from the cell lines listed in FIG. 3. The normal VIPR2 transcript was detected in all samples. No larger product corresponding to the predicted mutant transcript were observed in any sample. A truncated product was observed in one of the triplication carriers (05C43079). Sequencing of this transcript revealed an aberrantly spliced exon 3 and the creation of a premature translation termination site (data not shown). These results do not indicate that the duplication/triplication of VIPR2 results in a mutant transcript. If such a transcript is produced in patients with complex rearrangements of VIPR2, it is probably degraded by nonsense-mediated decay (NMD).

### 6.12. Clinical Description of 7q35.3 Duplication Carriers 02-0016 and 02-0135

Both patients were from a family triad sample collected from the US⁵. All patients in the sample participated in a formal diagnostic interview conducted by an experienced, licensed clinician specially trained in the use of the Diagnostic Interview for Genetic Studies (DIGS). Diagnostic criteria were applied as described in the Diagnostic and Statistical Manual 4 (DSM-IV). All diagnoses were confirmed by a senior licensed clinical psychologist.

Subject 02-0016.

Subject 02-0016 is male, age 44, of Norwegian descent. His diagnosis is schizoaffective disorder, depressed type, with onset at the age of 21. Family history: mother suffers from depression. Patient has never been married. He completed 4 years of college. He currently resides in a halfway house and attends a day treatment program. He is unemployed and receives disability. At age 16, he reported first feeling paranoid and having trouble in school. At age 21, he reported experiencing his first psychotic break and was hospitalized for 4 months. His symptoms primarily entailed paranoia. He also reported being depressed during his hospitalization. At age 22, he was hospitalized for a second time for suicidal thoughts, depression and feelings of hopelessness. At age 26, he experienced a relapse with prominent paranoid delusions. The patient had a history of hyperactivity prior to the age of 16. Developmentally, he reported maturation lag (i.e., growing up more slowly than other children). He also reported motor and vocal tics.

Subject 02-0135.

Subject 02-0135 is female, age 50, of Irish/German/English/Scottish descent. Her diagnosis is schizoaffective disorder, bipolar type, with onset at the age of 22. Family history: None known. Patient has never been married and her education level includes some college course work. She currently resides in a semi-independent group home and has been unemployed due to her psychiatric problems. Her primary psychiatric symptoms are paranoia and irritability. She began having significant psychiatric problems when she was 21 while she was away from home at college. She became aggressive and loud and started showing poor judgment. She has been hospitalized numerous times (at least 8) and her functioning has deteriorated steadily over the years. She has experienced at least 4 manic episodes, each lasting several months, with the first occurring when she was 25. The patient exhibited pressured speech, irritable mood, and grandiose ideation during each manic episode. She also reported obsessions with patterns but did not meet diagnostic criteria for OCD.

Developmentally, the patient has a history of delayed speech (did not speak words until the age of 3) and delayed puberty (did not menstruate until college). She lacked close friendships during childhood.

### 6.13. Association of 7q35.3 CNV with Psychiatric Phenotypes

Variable expressivity is often characteristic of pathogenic CNVs³⁰. The spectrum of psychiatric phenotypes associated with 7q36.3 duplications was evaluated by screening for these events in individuals with bipolar disorder or autism spectrum disorder (ASD). Microarray data was available for 2,777 patients from the Bipolar Genome Study (BiGS), for 996 ASD patients from the Autism Genome Project Consortium (AGP), and from unpublished analyses of 114 patients with ASD using the NimbleGen HD2 platform. Microduplications of 7q36.3 (>100 Kb in size) were detected in three of 1,110 (0.27%) of patients with ASD; compared with the controls described above, P=0.018. Microduplications at 7q36.3 were detected in two of 2,777 (0.07%) patients with bipolar disorder; compared with the controls, P=0.23. These results provide preliminary evidence that the clinical phenotypes associated with 7q36.3 duplications may include pediatric neurodevelopmental disorders such as autism, but do not include bipolar disorder. Also worthy of note, larger chromosomal abnormalities involving 7q have been reported in association with neurodevelopmental disorders, including deletions of 7q36-7qter^{36, 37} and duplications of 7q35-7qter.³⁸; a 550 Kb duplication of 7qter (of unknown clinical relevance) has been reported in a patient with neurofibromatosis³⁹.

### 6.14. Summary

These genetic data implicate the gene VIPR2. All variants contributing to the observed association at 7q36.3 overlap with this gene or lie within the gene-less subtelomeric region <89 kb from the transcriptional start site of VIPR2. In addition, partial triplications of VIPR2 involving only exons 3 and 4 were detected in four patients and zero controls. VIPR2 encodes the Vasoactive Intestinal Peptide (VIP) Receptor VPAC2, a G protein-coupled receptor that is expressed in variety of tissues including, in the brain, the suprachiasmatic nucleus, hippocampus, amygdala, and hypothalamus⁴⁰. VPAC2 binds VIP⁴¹, activates cyclic-AMP signaling and PKA, regulates synaptic transmission in the hippocampus⁴²⁻⁴⁴, and promotes the proliferation of neural progenitor cells in the dentate gyrus⁴⁵. Genetic studies in mouse have established that VIP signaling plays a role in learning and memory⁴⁶. VPAC2 also plays a role in sustaining normal circadian oscillations in the SCN^{47, 48}, and VIPR2-null⁴⁹ and VIPR2-overexpression⁵⁰ mice exhibit abnormal rhythms of rest and activity.

The observed expression patterns suggest that a variety of different genomic duplications can influence the transcription of VIPR2. Without being bound by theory, it is thought that duplications of 7q36 impact the regulation of VIPR2. Tandem duplication of regulatory sequences, for instance, could affect expression of the gene. Alternatively, the subtelomeric location of VIPR2 could be relevant to the mechanism. Intrinsic regulation of telomere structure and function often impacts the transcriptional regulation of adjacent genes, a phenomenon known as Telomere Position Effect (TPE)^{53, 54}. If VIPR2 is under such epigenetic regulation, any large tandem duplication of the subtelomeric region could potentially cause the gene to escape repression.

The present study implicates VIPR2 as a genetic susceptibility factor for schizophrenia. It has been shown that increased copy number of VIPR2 or the adjacent region is highly enriched in patients with schizophrenia. Moreover, disease mutations lead to increased VIPR2 transcription and VIP-induced cyclic-AMP signaling in patient cells. These results implicate VIP signaling as a molecular mechanism underlying schizophrenia. In light of the emerging roles of VIPR2 in the brain, the results described herein support the contention that the pathogenesis of schizophrenia, in some patients, involves the dysregulation of cellular processes such as adult neurogenesis and synaptic transmission and of the corresponding cognitive processes of learning and memory. Furthermore, in light of the brain expression patterns of VIPR2⁴⁰, the present results support the involvement of certain brain regions, such as hippocampus, amygdala and suprachiasmatic nucleus.

The link between VIPR2 duplications and schizophrenia may have significant implications for the development of molecular diagnostics and treatments for this disorder. Genetic testing for duplications of the 7q36 region could enable the early detection of a subtype of patients characterized by overexpression of VIPR2. Significant potential also exists for the development of therapeutics targeting this receptor. For instance, a selective antagonist of VPAC2R could have therapeutic potential in patients who carry duplications of the VIPR2 region. Peptide derivatives^{55, 56} and small molecules⁵⁷ have been identified that are selective VPAC2 inhibitors, and these pharmacological studies offer potential leads in the development of new drugs.

### REFERENCES

1. Walsh, T. et al. Rare Structural Variants Disrupt Multiple Genes in Neurodevelopmental Pathways in Schizophrenia. Science (2008).
2. Conrad, D. F., Andrews, T. D., Carter, N. P., Hurles, M. E. & Pritchard, J. K. A high-resolution survey of deletion polymorphism in the human genome. Nat Genet 38, 75-81 (2006).
3. McCarroll, S. A. et al. Common deletion polymorphisms in the human genome. Nat Genet 38, 86-92 (2006).
4. Brownstein, J. et al. Antisaccade performance is abnormal in schizophrenia patients but not in their biological relatives. Schizophr Res 63, 13-25 (2003).
5. DeLisi, L. E. et al. A genome-wide scan for linkage to chromosomal regions in 382 sibling pairs with schizophrenia or schizoaffective disorder. Am J Psychiatry 159, 803-12 (2002).
6. Mitchell, M. K., Gregersen, P. K., Johnson, S., Parsons, R. & Vlahov, D. The New York Cancer Project: rationale, organization, design, and baseline characteristics. J Urban Health 81, 301-10 (2004).
7. Talati, A., Fyer, A. J. & Weissman, M. M. A comparison between screened NIMH and clinically interviewed control samples on neuroticism and extraversion. Mol Psychiatry 13, 122-30 (2008).
8. Stone, J. L. et al. Rare chromosomal deletions and duplications increase risk of schizophrenia. Nature (2008).
9. Mahon, P. B. et al. Genome-wide linkage and follow-up association study of postpartum mood symptoms. Am J Psychiatry 166, 1229-37 (2009).
10. Li, C. & Hung Wong, W. Model-based analysis of oligonucleotide arrays: model validation, design issues and standard error application. Genome Biol 2, RESEARCH0032 (2001).
11. Diskin, S. J. et al. Adjustment of genomic waves in signal intensities from whole-genome SNP genotyping platforms. Nucleic Acids Res 36, e126 (2008).
12. Day, N., Hemmaplardh, A., Thurman, R. E., Stamatoyannopoulos, J. A. & Noble, W. S. Unsupervised segmentation of continuous genomic data. Bioinformatics 23, 1424-6 (2007).
13. Pique-Regi, R. et al. Sparse representation and Bayesian detection of genome copy number alterations from microarray data. Bioinformatics 24, 309-18 (2008).
14. McCarthy, S. E. et al. Microduplications of 16p11.2 are associated with schizophrenia. Nat Genet 41, 1223-7 (2009).
15. Williams, N. M. et al. Analysis of copy number variation using quantitative interspecies competitive PCR. Nucleic Acids Res 36, e112 (2008).
16. Mefford, H. C. et al. A method for rapid, targeted CNV genotyping identifies rare variants associated with neurocognitive disease. Genome Res 19, 1579-85 (2009).
17. McCarroll, S. A. et al. Integrated detection and population-genetic analysis of SNPs and copy number variation. Nat Genet 40, 1166-74 (2008).
18. Cummings, E. et al. Clinical symptomatology and the psychosis risk gene ZNF804A. Schizophr Res 122, 273-5.
19. Liu, H. et al. Genetic variation in the 22q11 locus and susceptibility to schizophrenia. Proc Natl Acad Sci USA 99, 16859-64 (2002).
20. Wang, K. et al. Common genetic variants on 5p14.1 associate with autism spectrum disorders. Nature (2009).
21. Zhang, D. et al. Singleton deletions throughout the genome increase risk of bipolar disorder. Mol. Psychiatry 14, 376-380 (2009).
22. Walsh, T. et al. Rare Structural Variants Disrupt Multiple Genes in Neurodevelopmental Pathways in Schizophrenia. Science (2008).
23. Stone, J. L. et al. Rare chromosomal deletions and duplications increase risk of schizophrenia. Nature (2008).
24. Stefansson, H. et al. Large recurrent microdeletions associated with schizophrenia. Nature (2008).
25. Xu, B. et al. Strong association of de novo copy number mutations with sporadic schizophrenia. Nat Genet 40, 880-5 (2008).
26. Sebat, J., Levy, D. L. & McCarthy, S. E. Rare structural variants in schizophrenia: one disorder, multiple mutations; one mutation, multiple disorders. Trends Genet 25, 528-35 (2009).
27. McClellan, J. & King, M. C. Genetic heterogeneity in human disease. Cell 141, 210-7 (2010).
28. Mulle, J. G. et al. Microdeletions of 3q29 confer high risk for schizophrenia. Am J Hum Genet 87, 229-36 (2010).
29. Karayiorgou, M. et al. Schizophrenia susceptibility associated with interstitial deletions of chromosome 2201. Proc Natl Acad Sci USA 92, 7612-6 (1995),
30. McCarthy, S. E. et al. Microduplications of 16p11.2 are associated with schizophrenia. Nat Genet 41, 1223-7 (2009).
31. Szatmari, P. et al. Mapping autism risk loci using genetic linkage and chromosomal rearrangements. Nat Genet 39, 319-28 (2007).
32. Kirov, G. et al. Comparative genome hybridization suggests a role for NRXN1 and APBA2 in schizophrenia. Hum Mol Genet 17, 458-65 (2008).
33. Rujescu, D. et al. Disruption of the neurexin 1 gene is associated with schizophrenia. Hum Mol Genet (2008).
34. Wang, K. et al. Common genetic variants on 5p14.1 associate with autism spectrum disorders. Nature (2009).
35. Zhang, D. et al. Singleton deletions throughout the genome increase risk of bipolar disorder. Mol Psychiatry (2008).
36. Tyson, C. et al. Submicroscopic deletions and duplications in individuals with intellectual disability detected by array-CGH. Am J Med Genet A 139, 173-85 (2005).
37. Wu, Y. et al. Submicroscopic subtelomeric aberrations in Chinese patients with unexplained developmental delay/mental retardation. BMC Med Genet 11, 72 (2010).
38. Morava, E. et al. Small inherited terminal duplication of 7q with hydrocephalus, cleft palate, joint contractures, and severe hypotonia. Clin Dysmorphol 12, 123-7 (2003).
39. Bartsch, O. et al. Two independent chromosomal rearrangements, a very small (550 kb) duplication of the 7q subtelomeric region and an atypical 17q11.2 (NF1) microdeletion, in a girl with neurofibromatosis. Cytogenet Genome Res 119, 158-64 (2007).
40. Sheward, W. J., Lutz, E. M. & Harmar, A. J. The distribution of vasoactive intestinal peptide2 receptor messenger RNA in the rat brain and pituitary gland as assessed by in situ hybridization. Neuroscience 67, 409-18 (1995).
41. Fahrenkrug, J. Transmitter role of vasoactive intestinal peptide. Pharmacol Toxicol 72, 354-63 (1993).
42. Yang, K. et al. Vasoactive intestinal peptide acts via multiple signal pathways to regulate hippocampal NMDA receptors and synaptic transmission. Hippocampus 19, 779-89 (2009).
43. Waschek, J. A. Vasoactive intestinal peptide: an important trophic factor and developmental regulator? Dev Neurosci 17, 1-7 (1995).
44. Hill, J. M. Vasoactive intestinal peptide in neurodevelopmental disorders: therapeutic potential. Curr Pharm Des 13, 1079-89 (2007).
45. Zaben, M. et al. The neurotransmitter VIP expands the pool of symmetrically dividing postnatal dentate gyrus precursors via VPAC2 receptors or directs them toward a neuronal fate via VPAC1 receptors. Stem Cells 27, 2539-51 (2009).
46. Chaudhury, D., Loh, D. H., Dragich, J. M., Hagopian, A. & Colwell, C. S. Select cognitive deficits in vasoactive intestinal peptide deficient mice. BMC Neurosci 9, 63 (2008).
47. Brown, T. M., Colwell, C. S., Waschek, J. A. & Piggins, H. D. Disrupted neuronal activity rhythms in the suprachiasmatic nuclei of vasoactive intestinal polypeptide-deficient mice. J Neurophysiol 97, 2553-8 (2007).
48. Vosko, A. M., Schroeder, A., Loh, D. H. & Colwell, C. S. Vasoactive intestinal peptide and the mammalian circadian system. Gen Comp Endocrinol 152, 165-75 (2007).
49. Harmar, A. J. et al. The VPAC (2) receptor is essential for circadian function in the mouse suprachiasmatic nuclei. Cell 109, 497-508 (2002).
50. Underhill, P. A. et al. Y chromosome sequence variation and the history of human populations. Nat Genet 26, 358-61 (2000).
51. Millar, J. K. et al. DISC1 and PDE4B are interacting genetic factors in schizophrenia that regulate cAMP signaling. Science 310, 1187-91 (2005).
52. Turetsky, B. I. & Moberg, P. J. An odor-specific threshold deficit implicates abnormal intracellular cyclic AMP signaling in schizophrenia. Am J Psychiatry 166, 226-33 (2009).
53. Gottschling, D. E., Aparicio, O. M., Billington, B. L. & Zakian, V. A. Position effect at S. cerevisiae telomeres: reversible repression of Pol II transcription. Cell 63, 751-62 (1990).
54. Koering, C. E. et al. Human telomeric position effect is determined by chromosomal context and telomeric chromatin integrity. EMBO Rep 3, 1055-61 (2002).
55. Moreno, D. et al. Development of selective agonists and antagonists for the human vasoactive intestinal polypeptide VPAC (2) receptor. Peptides 21, 1543-9 (2000).
56. Pan, C. Q., Hamren, S., Roczniak, S., Tom, I. & DeRome, M. Generation of PEGylated VPAC1-selective antagonists that inhibit proliferation of a lung cancer cell line. Peptides 29, 479-86 (2008).
57. Chu, A., Caldwell, J. S. & Chen, Y. A. Identification and characterization of a small molecule antagonist of human VPAC (2) receptor. Mol Pharmacol 77, 95-101 (2009).
58. Braun, M. M., Farag-El-Massah, S., Xu, K. & Cote, T. R. Emergence of orphan drugs in the United States: a quantitative assessment of the first 25 years. Nat Rev Drug Discov 9, 519-22 (2010).

TABLE 1
Events, Odds ratios, and Exact Conditional (EC) P-values listed here correspond to the peak of association.
Empirical P-values for the entire target region were then computed
based on permutation of case and control labels. The minimal threshold for statistical
significance after Bonferroni correction for the 114 loci tested was empirical P < 4.4 × 10 − 4.
When the number of controls in the secondary sample was 0, Haldane correction
(adding 0.5 to each cell of table) was applied in order to get a finite OR (*).
All genes overlapping with the target region are listed or the closest gene within 100 kb (**).

| | | | | Primary Sample | |
|---|---|---|---|---|---|
| Region (hg18) | Genes | Band | Type | Cases | Ctrls |
| chr22:19786712-19795854 | BCR | 22q11.2 | del | 2 | 0 |
| chr7:158731401-158810016 | VIPR2** | 7q36.3 | dup | 2 | 0 |
| chr16:29569647-30209382 | 28 genes | 16p11.2 | dup | 4 | 0 |
| chr15:29694064-29705665 | OTUD7A | 15q13.3 | del | 2 | 0 |
| chr7:158448321-158605936 | VIPR2, BC042556 | 7q36.3 | dup | 2 | 0 |
| chr15:28881608-28991107 | MTMR15 | 15q13.3 | del | 2 | 0 |
| chr3:196826549-196872080 | CR597873, SDHALP2 | 3q29 | dup | 2 | 0 |
| chr6:162835583-162997592 | PARK2 | 6q26 | dup | 2 | 0 |

| | Secondary Sample | | | | Permuation |
|---|---|---|---|---|---|
| Region (hg18) | Cases | Ctrls | Peak odds ratio | Peak P-value | P-value |
| chr22:19786712-19795854 | 22 | 0 | *14.21 [4.24, Inf] | 2.40E−06 | <5.00E−6 |
| chr7:158731401-158810016 | 18 | 1 | 16.41 [3.11, Inf] | 8.39E−05 | 4.00E−05 |
| chr16:29569647-30209382 | 18 | 1 | 16.14 [3.06, Inf] | 0.000097 | 0.0001 |
| chr15:29694064-29705665 | 16 | 1 | 14.94 [2.80, Inf] | 0.00023 | 0.00016 |
| chr7:158448321-158605936 | 12 | 0 | *8.26 [2.36, Inf] | 0.00086 | 0.0007 |
| chr15:28881608-28991107 | 16 | 1 | 14.94 [2.80, Inf] | 0.00023 | 0.001 |
| chr3:196826549-196872080 | 8 | 0 | *5.65 [1.56, Inf] | 0.01 | 0.005 |
| chr6:162835583-162997592 | 6 | 0 | *4.41 [1.17, Inf] | 0.03 | 0.044 |

TABLE 2
Breakdown of Samples Used in the Primary and Secondary Cohorts.

| | | Cases | | Controls | | Refer- |
|---|---|---|---|---|---|---|
| Dataset | Sample source | Initial | Filtered | Initial | Filtered | ences |
| Primary | U. of Washington | 163 | 149 | — | — | 1 |
| | Harvard | 389 | 331 | — | — | 5 |
| | Trinity College | 54 | 45 | — | — | 18 |
| | Dublin | | | | | |
| | McLean Hospital | 203 | 185 | — | — | 4 |
| | Columbia | 96 | 89 | — | — | 19 |
| | University | | | | | |
| | Coriell | 3 | 3 | — | — | |
| | NYCP | — | — | 287 | 222 | 6 |
| | HapMap | — | — | 179 | 164 | |
| | NIMH | — | — | 382 | 356 | 7 |
| | Total | 908 | 802 | 848 | 742 | |
| Secondary | MGS | 4,195 | 4,097 | 3,804 | 3,449 | 20 |
| | BiGS | — | — | 429 | 59 | 21 |
| | ISC | 3,391 | 3,391 | 3,181 | 3,181 | 8 |
| | Total | 7,586 | 7,488 | 7,414 | 6,689 | |
| Total | | 8,494 | 8,290 | 8,262 | 7,431 | |

TABLE 3
114 regions of interest defined in the primary dataset and their statistical association with schizophrenia estimated in
the secondary dataset.

| | | | | | | Region | Region | | | |
|---|---|---|---|---|---|---|---|---|---|---|
| | Region | Region | CNV | Primary | Primary | Secondary | Secondary | Region EC | | Permutation |
| Chrom | Start | End | Type | Cases | Controls | Cases | Controls | Odds Ratio | | P-value |
| 22 | 19,786,712 | 19,795,854 | del | 2 | 0 | 22 | 0 | * 14.21 | [3.55, 91.29] | <5.00E-6 |
| | 158,731,40 | | | | | | | | | |
| 7 | 1 | 158,810,016 | dup | 2 | 0 | 18 | 1 | 16.41 | [2.59, 682.17] | 4.00E-5 |
| 16 | 29,569,647 | 30,209,382 | dup | 4 | 0 | 20 | 5 | 3.58 | [1.30, 12.3] | 0.0001 |
| 15 | 29,694,064 | 29,705,665 | del | 2 | 0 | 16 | 1 | 14.94 | [2.3, 624.90] | 0.00016 |
| | 158,448,32 | | | | | | | | | |
| 7 | 1 | 158,605,936 | dup | 2 | 0 | 14 | 0 | * 9.37 | [2.25, 61.34] | 0.0007 |
| 15 | 28,881,608 | 28,991,107 | del | 2 | 0 | 16 | 2 | 7.47 | [1.76, 67.09] | 0.001005 |
| | 196,826,54 | | | | | | | | | |
| 3 | 9 | 196,872,080 | dup | 2 | 0 | 8 | 0 | * 5.65 | [1.27, 38.26] | 0.00498 |
| | 162,835,58 | | | | | | | | | |
| 6 | 3 | 162,997,592 | dup | 2 | 0 | 15 | 5 | 2.69 | [0.93, 9.49] | 0.0437 |
| 18 | 735,462 | 758,567 | dup | 2 | 0 | 4 | 0 | * 3.17 | [0.63, 22.76] | 0.07998 |
| | 162,613,14 | | | | | | | | | |
| 6 | 8 | 162,639,295 | dup | 2 | 0 | 13 | 8 | 1.46 | [0.56, 4.06] | 0.11811 |
| 19 | 63,541,593 | 63,717,416 | dup | 2 | 0 | 3 | 0 | * 2.68 | [0.50, 19.79] | 0.13852 |
| | 151,354,13 | | | | | | | | | |
| 7 | 7 | 151,531,740 | dup | 2 | 0 | 7 | 5 | 1.25 | [0.34, 5.01] | 0.14063 |
| | 242,596,39 | | | | | | | | | |
| 2 | 9 | 242,602,165 | del | 3 | 0 | 23 | 11 | 1.57 | [0.73, 3.58] | 0.14493 |
| | 131,933,96 | | | | | | | | | |
| 2 | 6 | 132,309,530 | dup | 2 | 0 | 5 | 1 | 4.50 | [0.50, 212.84] | 0.15642 |
| 15 | 22,076,912 | 22,222,014 | dup | 3 | 0 | 3 | 1 | 2.45 | [0.20, 129.22] | 0.15804 |
| | 104,190,79 | | | | | | | | | |
| 5 | 5 | 104,395,803 | del | 3 | 0 | 14 | 9 | 1.28 | [0.51, 3.35] | 0.15964 |
| 18 | 531,983 | 619,445 | dup | 2 | 0 | 6 | 2 | 2.62 | [0.47, 26.63] | 0.193 |
| 1 | 830,520 | 1,018,317 | del | 2 | 0 | 3 | 0 | * 2.34 | [0.44, 17.30] | 0.2013 |
| | 126,160,63 | | | | | | | | | |
| 2 | 0 | 126,207,714 | dup | 2 | 0 | 3 | 1 | 2.61 | [0.21, 137.28] | 0.21504 |
| 9 | 6,610,209 | 6,725,087 | dup | 2 | 0 | 11 | 5 | 1.99 | [0.64, 7.30] | 0.24002 |
| 17 | 21,445,191 | 21,476,126 | del | 3 | 0 | 2 | 0 | * 2.07 | [0.35, 15.96] | 0.26684 |
| 15 | 28,157,840 | 28,160,085 | del | 2 | 0 | 2 | 0 | * 2.06 | [0.35, 15.91] | 0.26721 |
| 7 | 5,791,341 | 5,898,394 | dup | 2 | 0 | 2 | 0 | *2.06 | [0.35, 15.87] | 0.26856 |
| | 110,665,42 | | | | | | | | | |
| 12 | 9 | 110,797,867 | dup | 2 | 0 | 19 | 15 | 1.16 | [0.56, 2.46] | 0.29414 |
| | 169,325,66 | | | | | | | | | |
| 6 | 1 | 169,540,112 | dup | 2 | 0 | 2 | 0 | * 1.96 | [0.33, 15.12] | 0.30058 |
| 8 | 4,036,835 | 4,258,540 | del | 3 | 0 | 3 | 2 | 1.26 | [0.14, 15.17] | 0.34208 |
| 2 | 45,262,920 | 45,829,157 | dup | 2 | 0 | 8 | 9 | 0.83 | [0.28, 2.43] | 0.34929 |
| 16 | 16,407,337 | 16,436,955 | del | 2 | 0 | 6 | 6 | 0.91 | [0.24, 3.41] | 0.36209 |
| 2 | 86,136,449 | 86,363,736 | dup | 2 | 0 | 9 | 6 | 1.40 | [0.45, 4.80] | 0.37314 |
| 4 | 63,267,552 | 63,351,723 | dup | 2 | 0 | 3 | 1 | 2.45 | [0.20, 129.22] | 0.39475 |
| | 153,240,84 | | | | | | | | | |
| 7 | 1 | 153,285,108 | dup | 2 | 0 | 5 | 3 | 1.56 | [0.30, 10.03] | 0.40339 |
| 5 | 37,323,428 | 37,627,559 | dup | 2 | 0 | 5 | 4 | 1.10 | [0.24, 5.56] | 0.4175 |
| 11 | 51,094,058 | 51,213,846 | dup | 2 | 0 | 15 | 15 | 0.88 | [0.40, 1.94] | 0.42744 |
| | 107,093,22 | | | | | | | | | |
| 10 | 5 | 107,306,521 | dup | 2 | 0 | 10 | 7 | 1.31 | [0.45, 4.07] | 0.44353 |
| 4 | 63,360,291 | 63,817,276 | dup | 2 | 0 | 4 | 3 | 1.11 | [0.19, 7.58] | 0.45086 |
| 19 | 59,253,486 | 59,409,048 | dup | 5 | 0 | 1 | 0 | * 1.46 | [0.21, 12.08] | 0.51261 |
| 11 | 72,112,160 | 72,234,378 | dup | 2 | 0 | 1 | 0 | * 1.46 | [0.21, 12.08] | 0.51474 |
| | | 242,400,85 | | | | | | | | |
| 2 | 3 | 242,492,158 | dup | 2 | 0 | 1 | 0 | * 1.46 | [0.21, 12.08] | 0.51525 |
| 17 | 75,291,024 | 75,436,783 | dup | 3 | 0 | 1 | 1 | 0.94 | [0.01, 73.62] | 0.5154 |
| 7 | 39,966,247 | 40,040,203 | dup | 2 | 0 | 1 | 1 | 0.94 | [0.01, 73.62] | 0.51705 |
| | 176,659,95 | | | | | | | | | |
| 5 | 1 | 176,879,167 | dup | 3 | 0 | 1 | 0 | * 1.45 | [0.21, 12.04] | 0.51713 |
| 11 | 66,760,906 | 67,047,142 | dup | 5 | 0 | 1 | 0 | * 1.46 | [0.21, 12.08] | 0.51724 |
| 17 | 21,246,527 | 21,289,471 | del | 3 | 0 | 1 | 0 | * 1.46 | [0.21, 12.08] | 0.51732 |
| | 138,054,38 | | | | | | | | | |
| 8 | 0 | 138,179,533 | del | 2 | 0 | 1 | 0 | * 1.46 | [0.21, 12.08] | 0.51842 |
| 12 | 6,754,237 | 6,946,504 | dup | 2 | 0 | 1 | 0 | * 1.45 | [0.21, 12.04] | 0.51878 |
| | 142,557,94 | | | | | | | | | |
| 1 | 8 | 142,697,860 | dup | 4 | 0 | 1 | 0 | * 1.45 | [0.21, 12.04] | 0.51915 |
| | 242,254,43 | | | | | | | | | |
| 2 | 4 | 242,292,729 | dup | 2 | 0 | 1 | 1 | 0.93 | [0.01, 72.66] | 0.52174 |
| 9 | 65,984,769 | 65,988,928 | del | 2 | 0 | 2 | 1 | 1.88 | [0.10, 110.68] | 0.5229 |
| 6 | 31,461,909 | 31,569,115 | del | 3 | 0 | 3 | 4 | 0.69 | [0.10, 4.11] | 0.52782 |
| | 142,803,84 | | | | | | | | | |
| 1 | 9 | 143,666,594 | dup | 8 | 0 | 9 | 8 | 0.91 | [0.31, 2.72] | 0.53701 |
| 12 | 20,899,786 | 21,300,773 | del | 3 | 0 | 4 | 3 | 1.24 | [0.21, 8.45] | 0.61408 |
| 22 | 35,043,725 | 35,197,506 | dup | 2 | 0 | 7 | 4 | 1.27 | [0.32, 5.96] | 0.63036 |
| 14 | 42,042,963 | 42,084,074 | del | 2 | 0 | 5 | 5 | 0.93 | [0.21, 4.06] | 0.66535 |
| 14 | 42,893,411 | 42,894,562 | dup | 2 | 0 | 7 | 7 | 0.91 | [0.27, 3.05] | 0.68996 |
| 15 | 19,927,930 | 21,022,680 | dup | 4 | 0 | 19 | 16 | 1.03 | [0.50, 2.14] | 0.70343 |
| 19 | 7,057,638 | 7,059,257 | dup | 2 | 0 | 16 | 15 | 0.97 | [0.45, 2.12] | 0.70648 |
| | 117,170,81 | | | | | | | | | |
| 7 | 4 | 118,949,288 | dup | 2 | 0 | 2 | 3 | 0.62 | [0.05, 5.45] | 0.74224 |
| 5 | 104,569,44 | 104,571,354 | del | 2 | 0 | 3 | 3 | 0.89 | [0.12, 6.66] | 0.75353 |
| | 7 | | | | | | | | | |
| 9 | 44,600,214 | 44,801,127 | dup | 3 | 0 | 1 | 1 | 0.94 | [0.01, 73.62] | 0.76595 |
| 5 | 1,368,542 | 1,580,687 | dup | 2 | 0 | 1 | 1 | 0.93 | [0.01, 73.12] | 0.76691 |
| 16 | 79,759,172 | 79,861,019 | dup | 2 | 0 | 1 | 2 | 0.46 | [0.01, 8.93] | 0.76986 |
| 9 | 39,706,964 | 39,799,828 | dup | 2 | 0 | 6 | 7 | 0.80 | [0.22, 2.80] | 0.80062 |
| 16 | 22,351,008 | 22,358,429 | dup | 4 | 0 | 9 | 11 | 0.74 | [0.27, 1.98] | 0.81115 |
| 7 | 62,471,393 | 62,737,937 | dup | 2 | 0 | 2 | 4 | 0.40 | [0.04, 2.84] | 0.82684 |
| 9 | 41,749,504 | 44,184,455 | dup | 4 | 0 | 4 | 10 | 0.37 | [0.09, 1.30] | 0.84919 |
| 8 | 7,982,049 | 8,185,108 | dup | 8 | 0 | 9 | 12 | 0.69 | [0.26, 1.78] | 0.86548 |
| | 102,628,52 | | | | | | | | | |
| 1 | 0 | 102,630,703 | del | 3 | 0 | 4 | 6 | 0.60 | [0.12, 2.54] | 0.86755 |
| | 116,122,97 | | | | | | | | | |
| 9 | 0 | 116,233,146 | dup | 2 | 0 | 1 | 2 | 0.46 | [0.01, 8.90] | 0.88862 |
| 9 | 30,428,790 | 30,551,803 | del | 2 | 0 | 9 | 12 | 0.68 | [0.25, 1.76] | 0.89612 |
| | 112,091,19 | | | | | | | | | |
| 2 | 3 | 112,193,141 | dup | 2 | 0 | 1 | 4 | 0.23 | [0, 2.36] | 0.916 |
| 1 | 73,562,153 | 73,569,411 | del | 2 | 0 | 1 | 2 | 0.39 | [0.01, 7.51] | 0.91675 |
| | 187,610,38 | | | | | | | | | |
| 1 | 3 | 187,693,945 | del | 3 | 0 | 3 | 5 | 0.50 | [0.08, 2.60] | 0.93501 |
| 15 | 20,199,291 | 20,307,804 | del | 3 | 0 | 15 | 18 | 0.72 | [0.34, 1.52] | 0.9372 |
| 16 | 18,207,840 | 18,285,452 | del | 2 | 0 | 1 | 3 | 0.31 | [0.01, 3.88] | 0.94543 |
| 13 | 63,157,506 | 63,273,479 | del | 5 | 0 | 10 | 17 | 0.54 | [0.22, 1.25] | 0.95314 |
| 5 | 12,578,860 | 12,716,534 | del | 2 | 0 | 2 | 5 | 0.33 | [0.03, 2.02] | 0.96361 |
| 7 | 29,635,150 | 29,754,584 | dup | 3 | 0 | 6 | 16 | 0.35 | [0.11, 0.94] | 0.978 |
| 13 | 42,447,865 | 42,586,103 | dup | 2 | 0 | 4 | 12 | 0.30 | [0.07, 0.99] | 0.9862 |
| | 180,149,57 | | | | | | | | | |
| 5 | 5 | 180,151,123 | dup | 2 | 0 | 1 | 5 | 0.19 | [0, 1.67] | 0.98747 |
| 5 | 180,044,59 | 180,045,767 | dup | 2 | 0 | 1 | 6 | 0.14 | [0, 1.18] | 0.9955 |
| | 5 | | | | | | | | | |
| 2 | 96,014,562 | 96,054,628 | dup | 2 | 0 | 0 | 1 | 0 | [0, 36.59] | 1 |
| | 233,024,51 | | | | | | | | | |
| 2 | 7 | 233,026,305 | del | 3 | 0 | 0 | 2 | 0 | [0, 4.93] | 1 |
| | 162,099,88 | | | | | | | | | |
| 4 | 1 | 162,222,538 | dup | 2 | 0 | 0 | 2 | 0 | [0, 4.99] | 1 |
| | 159,372,92 | | | | | | | | | |
| 6 | 0 | 159,492,244 | dup | 2 | 0 | 0 | 1 | 0 | [0, 36.11] | 1 |
| 7 | 66,095,737 | 66,399,480 | dup | 3 | 0 | 0 | 1 | 0 | [0, 36.11] | 1 |
| 9 | 40,823,291 | 41,560,441 | dup | 3 | 0 | 0 | 2 | 0 | [0, 4.99] | 1 |
| | 129,598,75 | | | | | | | | | |
| 9 | 5 | 129,780,195 | dup | 2 | 0 | 0 | 1 | 0 | [0, 36.11] | 1 |
| 16 | 18,689,806 | 18,697,579 | del | 2 | 0 | 0 | 2 | 0 | [0, 4.99] | 1 |

| | | | | | | | | |
|---|---|---|---|---|---|---|---|---|
| 1 | 1,161,232 | 1,266,721 | del | 2 | 0 | 0 | 0 | ** |
| 1 | 83,727,256 | 83,730,783 | del | 2 | 0 | 0 | 0 | ** |
| | 120,437,23 | | | | | | | |
| 1 | 7 | 120,490,996 | dup | 2 | 0 | 0 | 0 | ** |
| | 147,893,56 | | | | | | | |
| 1 | 2 | 147,905,665 | dup | 3 | 0 | 0 | 0 | ** |
| | 194,977,37 | | | | | | | |
| 1 | 0 | 195,180,249 | dup | 2 | 0 | 0 | 0 | ** |
| | 168,131,39 | | | | | | | |
| 2 | 6 | 168,247,871 | del | 2 | 0 | 0 | 0 | ** |
| | 211,798,51 | | | | | | | |
| 2 | 6 | 212,095,884 | del | 2 | 0 | 0 | 0 | ** |
| 3 | 12,950,276 | 13,031,569 | dup | 2 | 0 | 0 | 0 | ** |
| | 127,437,77 | | | | | | | |
| 3 | 0 | 127,460,707 | dup | 2 | 0 | 0 | 0 | ** |
| 3 | 176,153,28 | 176,565,692 | dup | 4 | 0 | 0 | 0 | ** |
| | 6 | | | | | | | |
| 4 | 8,555,340 | 8,701,803 | dup | 2 | 0 | 0 | 0 | ** |
| 4 | 70,057,584 | 70,063,155 | del | 2 | 0 | 0 | 0 | ** |
| 5 | 69,417,245 | 69,427,940 | del | 6 | 0 | 0 | 0 | ** |
| 5 | 70,426,301 | 70,572,367 | del | 2 | 0 | 0 | 0 | ** |
| 7 | 71,966,390 | 71,980,843 | dup | 2 | 0 | 0 | 0 | ** |
| | 136,504,21 | | | | | | | |
| 9 | 0 | 136,509,727 | del | 3 | 0 | 0 | 0 | ** |
| | 138,285,12 | | | | | | | |
| 9 | 2 | 138,395,102 | del | 2 | 0 | 0 | 0 | ** |
| | 126,264,77 | | | | | | | |
| 10 | 6 | 126,426,935 | dup | 4 | 0 | 0 | 0 | ** |
| 12 | 62,198,105 | 62,432,942 | del | 6 | 0 | 0 | 0 | ** |
| 13 | 85,595,764 | 85,711,152 | dup | 2 | 0 | 0 | 0 | ** |
| 17 | 43,108,368 | 43,144,482 | dup | 2 | 0 | 0 | 0 | ** |
| 19 | 7,429,361 | 7,530,268 | dup | 2 | 0 | 0 | 0 | ** |
| 19 | 48,194,812 | 48,225,405 | del | 2 | 0 | 0 | 0 | ** |
| 20 | 45,767,509 | 45,872,697 | dup | 2 | 0 | 0 | 0 | ** |
| 22 | 28,930,039 | 29,063,249 | dup | 3 | 0 | 0 | 0 | ** |
| 22 | 35,862,988 | 36,046,458 | dup | 2 | 0 | 0 | 0 | ** |

| | | | | | Peak | Peak | | | Peak EC |
|---|---|---|---|---|---|---|---|---|---|
| | Region | | | | Secondary | Secondary | Peak EC Odds | | P-value |
| Chrom | Start | Region End | Peak Start | Peak End | Cases | Controls | Ratio | | (one-sided) |
| 22 | 19,786,712 | 19,795,854 | 19,786,712 | 19,792,530 | 22 | 0 | * 14.21 | [4.24, Inf] | 2.40E-06 |
| 7 | 158,731,401 | 158,810,016 | 158,731,401 | 158,782,376 | 18 | 1 | 16.41 | [3.11, Inf] | 8.39E-05 |
| 16 | 29,569,647 | 30,209,382 | 29,997,384 | 30,011,876 | 18 | 1 | 16.14 | [3.06, Inf] | 0.000097 |
| 15 | 29,694,064 | 29,705,665 | 29,694,064 | 29,705,665 | 16 | 1 | 14.94 | [2.80, Inf] | 0.0002303 |
| 7 | 158,448,321 | 158,605,936 | 158,448,321 | 158,502,254 | 12 | 0 | * 8.26 | [2.36, Inf] | 0.0008554 |
| 15 | 28,881,608 | 28,991,107 | 28,936,449 | 28,991,107 | 16 | 1 | 14.94 | [2.80, Inf] | 0.0002303 |
| 3 | 196,826,549 | 196,872,080 | 196,868,311 | 196,872,080 | 8 | 0 | * 5.65 | [1.56, Inf] | 0.009763 |
| 6 | 162,835,583 | 162,997,592 | 162,976,775 | 162,979,293 | 6 | 0 | * 4.41 | [1.17, Inf] | 0.03035 |
| 18 | 735,462 | 758,567 | 735,462 | 745,862 | 4 | 0 | *3.17 | [0.79, Inf] | 0.09013366 |
| 6 | 162,613,148 | 162,639,295 | 162,613,148 | 162,632,337 | 12 | 5 | 2.14 | [0.81, Inf] | 0.1112766 |
| 19 | 63,541,593 | 63,717,416 | 63,641,391 | 63,678,611 | 3 | 0 | * 2.68 | [0.64, Inf] | 0.1380708 |
| 7 | 151,354,137 | 151,531,740 | 151,448,335 | 151,451,766 | 6 | 1 | 5.41 | [0.83, Inf] | 0.08171413 |
| 2 | 242,596,399 | 242,602,165 | 242,596,399 | 242,602,165 | 23 | 11 | 1.57 | [0.81, Inf] | 0.1451722 |
| 2 | 131,933,966 | 132,309,530 | 131,933,966 | 131,983,008 | 5 | 1 | 4.50 | [0.64, Inf] | 0.1368308 |
| 15 | 22,076,912 | 22,222,014 | 22,083,414 | 22,222,014 | 3 | 0 | *2.56 | [0.61, Inf] | 0.1508959 |
| 5 | 104,190,795 | 104,395,803 | 104,283,507 | 104,295,984 | 11 | 3 | 3.01 | [0.94, Inf] | 0.06344 |
| 18 | 531,983 | 619,445 | 533,131 | 548,354 | 6 | 2 | 2.62 | [0.58, Inf] | 0.1939171 |
| 1 | 830,520 | 1,018,317 | 890,962 | 1,018,317 | 3 | 0 | *2.34 | [0.56, Inf] | 0.1786365 |
| 2 | 126,160,630 | 126,207,714 | 126,168,390 | 126,207,714 | 3 | 0 | *2.56 | [0.61, Inf] | 0.1515399 |
| 9 | 6,610,209 | 6,725,087 | 6,702,314 | 6,705,049 | 8 | 3 | 2.37 | [0.69, Inf] | 0.1557891 |
| 17 | 21,445,191 | 21,476,126 | 21,445,191 | 21,459,694 | 2 | 0 | * 2.07 | [0.46, Inf] | 0.2273187 |
| 15 | 28,157,840 | 28,160,085 | 28,157,840 | 28,160,085 | 2 | 0 | * 2.06 | [0.46, Inf] | 0.2282553 |
| 7 | 5,791,341 | 5,898,394 | 5,846,784 | 5,898,394 | 2 | 0 | * 2.06 | [0.46, Inf] | 0.2291737 |
| 12 | 110,665,429 | 110,797,867 | 110,797,494 | 110,797,867 | 16 | 9 | 1.62 | [0.77, Inf] | 0.1653556 |
| 6 | 169,325,661 | 169,540,112 | 169,325,836 | 169,540,086 | 2 | 0 | * 1.96 | [0.44, Inf] | 0.2468666 |
| 8 | 4,036,835 | 4,258,540 | 4,128,796 | 4,131,148 | 2 | 0 | * 2.07 | [0.46, Inf] | 0.2273187 |
| 2 | 45,262,920 | 45,829,157 | 45,828,360 | 45,829,157 | 4 | 1 | 3.75 | [0.49, Inf] | 0.2080142 |
| 16 | 16,407,337 | 16,436,955 | 16,407,337 | 16,420,592 | 6 | 3 | 1.81 | [0.47, Inf] | 0.3070019 |
| 2 | 86,136,449 | 86,363,736 | 86,186,704 | 86,196,300 | 8 | 4 | 1.87 | [0.60, Inf] | 0.2278156 |
| 4 | 63,267,552 | 63,351,723 | 63,268,491 | 63,350,429 | 3 | 1 | 2.45 | [0.27, Inf] | 0.3925937 |
| 7 | 153,240,841 | 153,285,108 | 153,240,841 | 153,247,628 | 5 | 3 | 1.56 | [0.38, Inf] | 0.401487 |
| 5 | 37,323,428 | 37,627,559 | 37,409,530 | 37,462,901 | 5 | 2 | 2.17 | [0.45, Inf] | 0.2894983 |
| 11 | 51,094,058 | 51,213,846 | 51,185,351 | 51,193,650 | 14 | 9 | 1.35 | [0.62, Inf] | 0.3128137 |
| 10 | 107,093,225 | 107,306,521 | 107,093,225 | 107,100.981 | 6 | 3 | 1.81 | [0.47, Inf] | 0.3070019 |
| 4 | 63,360,291 | 63,817,276 | 63,362,225 | 63,641,655 | 3 | 1 | 2.45 | [0.27, Inf] | 0.3925937 |
| 19 | 59,253,486 | 59,409,048 | 59,372,574 | 59,409,048 | 1 | 0 | * 1.46 | [0.29, Inf] | 0.364769 |
| 11 | 72,112,160 | 72,234,378 | 72,112,160 | 72,225,335 | 1 | 0 | * 1.46 | [0.29, Inf] | 0.364769 |
| 2 | 242,400,853 | 242,492,158 | 242,420,084 | 242,492,158 | 1 | 0 | * 1.46 | [0.29, Inf] | 0.364769 |
| 17 | 75,291,024 | 75,436,783 | 75,291,024 | 75,434,447 | 1 | 0 | * 1.46 | [0.29, Inf] | 0.364769 |
| 7 | 39,966,247 | 40,040,203 | 39,966,247 | 39,975,097 | 1 | 0 | * 1.46 | [0.29, Inf] | 0.364769 |
| 5 | 176,659,951 | 176,879,167 | 176,659,951 | 176,879,167 | 1 | 0 | * 1.45 | [0.29, Inf] | 0.3660872 |
| 11 | 66,760,906 | 67,047,142 | 66,760,906 | 67,038,667 | 1 | 0 | * 1.46 | [0.29, Inf] | 0.364769 |
| 17 | 21,246,527 | 21,289,471 | 21,272,823 | 21,289,471 | 1 | 0 | * 1.46 | [0.29, Inf] | 0.364769 |
| 8 | 138,054,380 | 138,179,533 | 138,054,380 | 138,179,533 | 1 | 0 | * 1.46 | [0.29, Inf] | 0.364769 |
| 12 | 6,754,237 | 6,946,504 | 6,754,237 | 6,946,504 | 1 | 0 | * 1.45 | [0.29, Inf] | 0.3660872 |
| 1 | 142,557,948 | 142,697.860 | 142,557,948 | 142,697,860 | 1 | 0 | * 1.45 | [0.29, Inf] | 0.3660872 |
| 2 | 242,254,434 | 242,292,729 | 242,254,635 | 242,292,729 | 1 | 0 | * 1.45 | [0.29, Inf] | 0.3660872 |
| 9 | 65,984,769 | 65,988,928 | 65,984,769 | 65,988,928 | 2 | 1 | 1.88 | [0.15, Inf] | 0.5239608 |
| 6 | 31,461,909 | 31,569,115 | 31,461,909 | 31,467,627 | 3 | 1 | 2.78 | [0.31, Inf] | 0.3418555 |
| 1 | 142,803,849 | 143,666,594 | 143,183,331 | 143,183,343 | 6 | 2 | 2.21 | [0.49, Inf] | 0.2681109 |
| 12 | 20,899,786 | 21,300,773 | 21,228,225 | 21,273,886 | 4 | 3 | 1.24 | [0.27, Inf] | 0.5410493 |
| 22 | 35,043,725 | 35,197,506 | 35,046,248 | 35,059,131 | 3 | 1 | 2.28 | [0.25, Inf] | 0.4212802 |
| 14 | 42,042,963 | 42,084,074 | 42,042,963 | 42,084,074 | 5 | 5 | 0.93 | [0.27, Inf] | 0.6640784 |
| 14 | 42,893,411 | 42,894,562 | 42,893,566 | 42,894,562 | 7 | 7 | 0.91 | [0.33, Inf] | 0.6699138 |
| 15 | 19,927,930 | 21,022,680 | 20,232,428 | 20,232,602 | 15 | 11 | 1.18 | [0.57, Inf] | 0.4177382 |
| 19 | 7,057,638 | 7,059,257 | 7,057,724 | 7,059,050 | 7 | 6 | 1.08 | [0.37, Inf] | 0.5533932 |
| 7 | 117,170,814 | 118,949,288 | 118,947,665 | 118,949,288 | 2 | 1 | 1.87 | [0.15, Inf] | 0.5255947 |
| 5 | 104,569,447 | 104,571,354 | 104,571,030 | 104,571,354 | 3 | 3 | 0.89 | [0.16, Inf] | 0.709417 |
| 9 | 44,600,214 | 44,801,127 | 44,600,214 | 44,801,127 | 1 | 1 | 0.94 | [0.02, Inf] | 0.7657596 |
| 5 | 1,368,542 | 1,580,687 | 1,368,542 | 1,580,687 | 1 | 1 | 0.93 | [0.02, Inf] | 0.767307 |
| 16 | 79,759,172 | 79,861,019 | 79,850,232 | 79,861,019 | 1 | 1 | 0.93 | [0.02, Inf] | 0.7689284 |
| 9 | 39,706,964 | 39,799,828 | 39,706,964 | 39,768,632 | 6 | 7 | 0.80 | [0.27, Inf] | 0.7485114 |
| 16 | 22,351,008 | 22,358,429 | 22,351,008 | 22,358,429 | 9 | 11 | 0.74 | [0.32, Inf] | 0.8119028 |
| 7 | 62,471,393 | 62,737,937 | 62,546,960 | 62,737,937 | 1 | 0 | * 1.36 | [0.27, Inf] | 0.392528 |
| 9 | 41,749,504 | 44,184,455 | 42,014,171 | 43,117,876 | 1 | 0 | * 1.46 | [0.29, Inf] | 0.364769 |
| 8 | 7,982,049 | 8,185,108 | 8,183,342 | 8,183,409 | 2 | 1 | 1.85 | [0.14, Inf] | 0.5288696 |
| 1 | 102,628,520 | 102,630,703 | 102,628,520 | 102,630,703 | 4 | 6 | 0.60 | [0.16, Inf] | 0.8671121 |
| 9 | 116,122,970 | 116,233,146 | 116,122,970 | 116,233,146 | 1 | 2 | 0.46 | [0.02, Inf] | 0.8889577 |
| 9 | 30,428,790 | 30,551,803 | 30,428,790 | 30,502,185 | 8 | 11 | 0.67 | [0.27, Inf] | 0.8656363 |
| 2 | 112,091,193 | 112,193,141 | 112,191,939 | 112,193,141 | 1 | 2 | 0.47 | [0.02, Inf] | 0.8873895 |
| 1 | 73,562,153 | 73,569,411 | 73,562,153 | 73,569,411 | 1 | 2 | 0.39 | [0.01, Inf] | 0.91666 |
| 1 | 187,610,383 | 187,693,945 | 187,610,383 | 187,617,184 | 2 | 4 | 0.44 | [0.6, Inf] | 0.9159532 |
| 15 | 20,199,291 | 20,307,804 | 20,302,447 | 20,307,305 | 15 | 17 | 0.76 | [0.40, Inf] | 0.8257137 |
| 16 | 18,207,840 | 18,285,452 | 18,207,840 | 18,285,452 | 1 | 3 | 0.31 | [0.01, Inf] | 0.9455116 |
| 13 | 63,157,506 | 63,273,479 | 63,271,230 | 63,273,479 | 3 | 4 | 0.67 | [0.13, Inf] | 0.8159973 |
| 5 | 12,578,860 | 12,716,534 | 12,712,618 | 12,716,534 | 2 | 5 | 0.33 | [0.05, Inf] | 0.9633796 |
| 7 | 29,635,150 | 29,754,584 | 29,747,479 | 29,750,239 | 6 | 9 | 0.62 | [0.22, Inf] | 0.8768183 |
| 13 | 42,447,865 | 42,586,103 | 42,447,865 | 42,452,940 | 4 | 10 | 0.36 | [0.10, Inf] | 0.9826215 |
| 5 | 180,149,575 | 180,151,123 | 180,149,575 | 180,151,123 | 1 | 5 | 0.19 | [0.01, Inf] | 0.9873297 |
| 5 | 180,044,595 | 180,045,767 | 180,044,595 | 180,045,767 | 1 | 6 | 0.14 | [0.01, Inf] | 0.9955537 |
| 2 | 96,014,562 | 96,054,628 | 96,014,562 | 96,054,628 | 0 | 1 | 0 | [0.01, Inf] | 1 |
| 2 | 233,024,517 | 233,026,305 | 233,024,517 | 233,026,305 | 0 | 2 | 0 | [0.01, Inf] | 1 |
| 4 | 162,099,881 | 162,222,538 | 162,099,881 | 162,222,538 | 0 | 2 | 0 | [0.01, Inf] | 1 |
| 6 | 159,372,920 | 159,492,244 | 159,469,377 | 159,492,244 | 0 | 1 | 0 | [0.01, Inf] | 1 |
| 7 | 66,095,737 | 66,399,480 | 66,337,219 | 66,399,480 | 0 | 1 | 0 | [0.01, Inf] | 1 |
| 9 | 40,823,291 | 41,560,441 | 40,823,291 | 40,901,225 | 0 | 1 | 0 | [0.01, Inf] | 1 |
| 9 | 129,598,755 | 129,780,195 | 129,762,905 | 129,780,195 | 0 | 1 | 0 | [0.01, Inf] | 1 |
| 16 | 18,689,806 | 18,697,579 | 18,689,806 | 18,697,579 | 0 | 2 | 0 | [0.01, Inf] | 1 |

| | | |
|---|---|---|
| 1 | 1,161,232 | 1,266,721 |
| 1 | 83,727,256 | 83,730,783 |
| 1 | 120,437,237 | 120,490,996 |
| 1 | 147,893,562 | 147,905,665 |
| 1 | 194,977,370 | 195,180,249 |
| 2 | 168,131,396 | 168,247,871 |
| 2 | 211,798,516 | 212,095,884 |
| 3 | 12,950,276 | 13,031,569 |
| 3 | 127,437,770 | 127,460,707 |
| 3 | 176,153,286 | 176,565,692 |
| 4 | 8,555,340 | 8,701,803 |
| 4 | 70,057,584 | 70,063,155 |
| 5 | 69,417,245 | 69,427,940 |
| 5 | 70,426,301 | 70,572,367 |
| 7 | 71,966,390 | 71,980,843 |
| 9 | 136,504,210 | 136,509,727 |
| 9 | 138,285,122 | 138,395,102 |
| 10 | 126,264,776 | 126,426,935 |
| 12 | 62,198,105 | 62,432,942 |
| 13 | 85,595,764 | 85,711,152 |
| 17 | 43,108,368 | 43,144,482 |
| 19 | 7,429,361 | 7,530,268 |
| 19 | 48,194,812 | 48,225,405 |
| 20 | 45,767,509 | 45,872,697 |
| 22 | 28,930,039 | 29,063,249 |
| 22 | 35,86,988 | 36,046,458 |

* when the number of controls in the secondary sample was 0, Haldane correction (adding 0.5 to each table count) was applied in order to get a finite OR
** event did not exist in the secondary dataset

TABLE 4
Duplications and Triplications of 7q36.3 Detected in Primary, MGS and ISC Datasets. Genomic coordinates of
duplications and nested triplications are listed separately. Validation results are reported for all 16 events that were detected in
the primary and MGS datasets. One duplication in control sample 06C52730 failed validation by the Sequenom assay.

| | | | Age of | | | | | |
|---|---|---|---|---|---|---|---|---|
| Sample ID | CNV ID | Phenotype | Onset | Ethnicity | Sex | Dataset | Platform | Chrom |
| 02-0135 | — | Schizophrenia | 23 | Caucasian | F | Primary | NimbleGen HD2 | 7 |
| | | Catatonic | | | | | | |
| LW-102-04 | — | Schizophrenia | 12 | Hispanic | M | Primary | NimbleGen HD2 | 7 |
| 02-0016 | — | Schizophrenia | 21 | Caucasian | M | Primary | NimbleGen HD2 | 7 |
| D0024922 | — | Control | N/A | African-American | M | Primary | NimbleGen HD2 | 7 |
| 05C48386 | — | Schizophrenia | 35 | Caucasian | M | MGS | NimbleGen HD2 | 7 |
| 05C51123 | — | Schizophrenia | 25 | Caucasian | M | MGS | NimbleGen HD2 | 7 |
| 05C43079 | — | Schizophrenia | 16 | Caucasian | M | MGS | NimbleGen HD2 | 7 |
| 03C23250 | — | Schizophrenia | 31 | Caucasian | F | MGS | Affymetrix 6.0 | 7 |
| 03C23091 | — | Schizophrenia | 21 | Caucasian | M | MGS | NimbleGen HD2 | 7 |
| 00C02204 | — | Schizophrenia | — | Caucasian | M | MGS | NimbleGen HD2 | 7 |
| 05C48694 | — | Schizophrenia | 16 | Caucasian | M | MGS | NimbleGen HD2 | 7 |
| 02C13414 | — | Schizophrenia | 23 | African-American | M | MGS | NimbleGen HD2 | 7 |
| 00C02873 | — | Schizophrenia | 17 | African-American | M | MGS | NimbleGen HD2 | 7 |
| 05C44574 | — | Schizophrenia | 17 | Caucasian | M | MGS | NimbleGen HD2 | 7 |
| 05C46770 | — | Schizophrenia | 17 | Caucasian | M | MGS | NimbleGen HD2 | 7 |
| *06C52730 | — | Control | N/A | Caucasian | M | MGS | Affymetrix 6.0 | 7 |
| — | ISC-1492 | Schizophrenia | — | Caucasian | — | ISC | Affymetrix 5.0/6.0 | 7 |
| — | ISC-2139 | Schizophrenia | — | Caucasian | — | ISC | Affymetrix 5.0/6.0 | 7 |
| — | ISC-2475 | Schizophrenia | — | Caucasian | — | ISC | Affymetrix 5.0/6.0 | 7 |
| — | ISC-3320 | Schizophrenia | — | Caucasian | — | ISC | Affymetrix 5.0/6.0 | 7 |
| — | ISC-3496 | Schizophrenia | — | Caucasian | — | ISC | Affymetrix 5.0/6.0 | 7 |
| — | ISC-3465 | Schizophrenia | — | Caucasian | — | ISC | Affymetrix 5.0/6.0 | 7 |
| — | ISC-3423 | Schizophrenia | — | Caucasian | — | ISC | Affymetrix 5.0/6.0 | 7 |
| — | ISC-2540 | Schizophrenia | — | Caucasian | — | ISC | Affymetrix 5.0/6.0 | 7 |
| — | ISC-2620 | Schizophrenia | — | Caucasian | — | ISC | Affymetrix 5.0/6.0 | 7 |
| — | ISC-3446 | Schizophrenia | — | Caucasian | — | ISC | Affymetrix 5.0/6.0 | 7 |
| — | ISC-2570 | Schizophrenia | — | Caucasian | — | ISC | Affymetrix 5.0/6.0 | 7 |
| — | ISC-2574 | Schizophrenia | — | Caucasian | — | ISC | Affymetrix 5.0/6.0 | 7 |
| — | ISC-3435 | Schizophrenia | — | Caucasian | — | ISC | Affymetrix 5.0/6.0 | 7 |
| — | ISC-2595 | Schizophrenia | — | Caucasian | | ISC | Affymetrix 5.0/6.0 | 7 |
| — | ISC-3480 | Schizophrenia | — | Caucasian | | ISC | Affymetrix 5.0/6.0 | 7 |
| Sample ID | | | | | | | | Sequenom |
| ID | CNV ID | Dup.Start | Dup.End | Dup.Length | Tripl.Start | Tripl.End | Tripl.Length | Validation |
| 02-0135 | — | 152,249,238 | 158,820,241 | 6,571,003 | — | — | — | Confirmed |
| LW-102-04 | — | 158,448,322 | 158,651,373 | 203,051 | — | — | — | Confirmed |
| 02-0016 | — | 158,703,311 | 158,810,016 | 106,705 | — | — | — | Confirmed |
| D0024922 | — | 158,605,937 | 158,731,401 | 125,464 | — | — | — | Confirmed |
| 05C48386 | — | 158,400,168 | 158,782,022 | 381,854 | — | — | — | Confirmed |
| 05C51123 | — | 158,699,364 | 158,820,241 | 120,877 | — | — | — | Confirmed |
| 05C43079 | — | 158,322,402 | 158,652,547 | 330,145 | 158,543,969 | 158,614,169 | 70,200 | Confirmed |
| 03C23250 | — | 158,322,880 | 158,650,683 | 327,803 | 158,540,185 | 158,621,330 | 81,145 | Confirmed |
| 03C23091 | — | 158,307,494 | 158,650,321 | 342,827 | 158,396,852; | 158,456,333; | 59,481; 51,646 | Confirmed |
| | | | | | 158,562,524 | 158,614,169 | | Confirmed |
| 00C02204 | — | 158,265,451 | 158,652,547 | 387,096 | 158,550,023 | 158,614,169 | 64,146 | Confirmed |
| 05C48694 | — | 158,171,999 | 158,826,119 | 654,120 | — | — | — | Confirmed |
| 02C13414 | — | 158,589,435 | 158,820,241 | 230,806 | — | — | — | Confirmed |
| 00C02873 | — | 158,618,998 | 158,780,576 | 161,578 | 158,654,446 | 158,726,336 | 71,890 | Confirmed |
| 05C44574 | — | 158,275,536 | 158,779,706 | 504,170 | — | — | — | Confirmed |
| 05C46770 | — | 158,699,364 | 158,820,241 | 120,877 | — | — | — | Confirmed |
| *06C52730 | — | 158,694,581 | 158,819,753 | 125,172 | — | — | — | Not |
| | | | | | | | | Confirmed |
| — | ISC-1492 | 157,571,900 | 158,677,051 | 1,105,151 | — | — | — | — |
| — | ISC-2139 | 158,096,725 | 158,502,254 | 405,529 | — | — | — | — |
| — | ISC-2475 | 158,121,784 | 158,588,486 | 466,702 | — | — | — | — |
| — | ISC-3320 | 158,276,605 | 158,819,766 | 543,161 | — | — | — | — |
| — | ISC-3496 | 158,384,773 | 158,512,459 | 127,686 | — | — | — | — |
| — | ISC-3465 | 158,569,154 | 158,819,536 | 250,382 | — | — | — | — |
| — | ISC-3423 | 158,664,993 | 158,819,536 | 154,543 | — | — | — | — |
| — | ISC-2540 | 158,690,919 | 158,819,536 | 128,617 | — | — | — | — |
| — | ISC-2620 | 158,690,919 | 158,819,536 | 128,617 | — | — | — | — |
| — | ISC-3446 | 158,690,919 | 158,819,536 | 128,617 | — | — | — | — |
| — | ISC-2570 | 158,712,866 | 158,819,536 | 106,670 | — | — | — | — |
| — | ISC-2574 | 158,712,866 | 158,819,536 | 106,670 | — | — | — | — |
| — | ISC-3435 | 158,712,866 | 158,819,536 | 106,670 | — | — | — | — |
| — | ISC-2595 | 158,719,441 | 158,819,536 | 100,095 | — | — | — | — |
| — | ISC-3480 | 158,719,441 | 158,819,536 | 100,095 | — | — | — | — |

TABLE 5
Adaptive Confidence Ccore (CS) Thresholds for
a) NimbleGen HD2, and b) Affymetrix 6.0 Platforms.

| | |
|---|---|
| Number of probes | P-value threshold |

a

| | |
|---|---|
| 5-10 | 1.0 × 10⁻⁶ |
| 11-18 | 2.5 × 10⁻⁶ |
| 19-44 | 2.5 × 10⁻⁵ |
| 45-100 | 2.5 × 10⁻³ |
| 101-250 | 1.0 × 10⁻² |
| ≧251 | 2.5 × 10⁻⁴ |

b

| | |
|---|---|
| 16-23 | 1.0 × 10⁻⁶ |
| 24-32 | 2.5 × 10⁻⁵ |
| 33-44 | 2.5 × 10⁻⁴ |
| ≧45 | 5.0 × 10⁻² |

TABLE 6
Ancestry Breakdown of Samples Used in the Primary and
Secondary Cohorts.

| Dataset | Study | Platform | Ancestry | Cases | Control | Total |
|---|---|---|---|---|---|---|
| Primary | This | NimbleGen | African- | 48 | 192 | 240 |
| | study | HD2 | American | | | |
| | This | NimbleGen | Caucasian | 640 | 506 | 1,146 |
| | study | HD2 | | | | |
| | This | NimbleGen | Hispanic | 114 | 44 | 158 |
| | study | HD2 | | | | |
| Total | | | | 802 | 742 | 1,544 |
| Secondary | MGS | Affymetrix | African- | 1,319 | 936 | 2,255 |
| | | 6.0 | American | | | |
| | MGS | Affymetrix | Caucasian | 2,778 | 2,572 | 5,350 |
| | | 6.0 | | | | |
| | ISC | Affymetrix | Caucasian | 3,391 | 3,181 | 6,572 |
| | | 5.0, 6.0 | | | | |
| Total | | | | 7,488 | 6,689 | 14,177 |

TABLE 7
Evaluating the Sensitivity to Detect Common CNPs Based on
Concordance Between Segmentation Calls and CNP Genotypes
Obtained Using a Targeted Genotyping Method

| | | | | | | Case: | |
|---|---|---|---|---|---|---|---|
| | | Cases | | Controls | | Control | |
| | | Mean | | Mean | | Sens. | P- |
| | | Sens. | SE | Sens. | SE | Ratio | value |
| European | All | 0.72 | 0.0007 | 0.71 | 0.0007 | 1.01 | 0.1817 |
| | CNVs | | | | | | |
| | ≧100 kb | 0.99 | 0.0002 | 0.99 | 0.0003 | 1.00 | 0.8379 |
| | <100 kb | 0.62 | 0.0013 | 0.60 | 0.0016 | 1.03 | 0.0760 |
| Hispanic | All | 0.74 | 0.0015 | 0.69 | 0.0027 | 1.07 | 0.0382 |
| | CNVs | | | | | | |
| | ≧100 kb | 0.99 | 0.0001 | 1.00 | 0.0000 | 0.99 | 0.1625 |
| | <100 kb | 0.63 | 0.0034 | 0.54 | 0.0047 | 1.17 | 0.0072 |
| African- | All | 0.72 | 0.0038 | 0.73 | 0.0019 | 0.98 | 0.5533 |
| Am. | CNVs | | | | | | |
| | ≧100 kb | 0.99 | 0.0004 | 0.99 | 0.0002 | 1.00 | 0.8862 |
| | <100 kb | 0.60 | 0.0058 | 0.60 | 0.0047 | 1.00 | 0.9542 |
| Total | All | 0.72 | 0.0006 | 0.72 | 0.0007 | 1.01 | 0.2603 |
| | CNVs | | | | | | |
| | ≧100 kb | 0.99 | 0.0001 | 0.99 | 0.0002 | 1.00 | 0.9632 |
| | <100 kb | 0.62 | 0.0012 | 0.60 | 0.0016 | 1.04 | 0.0289 |
| European | All | 0.42 | 0.0005 | 0.42 | 0.0005 | 1.01 | 0.1856 |
| | CNVs | | | | | | |
| | ≧100 kb | 0.70 | 0.0020 | 0.70 | 0.0022 | 1.01 | 0.4177 |
| | <100 kb | 0.34 | 0.0006 | 0.34 | 0.0006 | 1.01 | 0.5101 |
| African- | All | 0.37 | 0.0007 | 0.39 | 0.0009 | 0.97 | 0.1234 |
| Am. | CNVs | | | | | | |
| | ≧100 kb | 0.86 | 0.0024 | 0.90 | 0.0019 | 0.96 | 0.0014 |
| | <100 kb | 0.26 | 0.0008 | 0.26 | 0.0010 | 1.00 | 0.8978 |
| Total | All | 0.41 | 0.0004 | 0.41 | 0.0004 | 1.00 | 0.7420 |
| | CNVs | | | | | | |
| | ≧100 kb | 0.75 | 0.0016 | 0.75 | 0.0018 | 1.00 | 0.7375 |
| | <100 kb | 0.32 | 0.0005 | 0.32 | 0.0005 | 0.99 | 0.6470 |

TABLE 8
Sequences of primers for CNV validation assays.

| | | | | Human | Chimp |
|---|---|---|---|---|---|
| Primer Name | Forward Primer | Reverse Primer | Extension Primer | Allele | Allele |
| 7qter-L | CAGATTTTATTAGGACAAAGC | GGGTAATTTTTCTCAATTCC | AGTAGGACAAAGCAGAACA | C | T |
| 7qter-R | GCATCTACTTCCAGTTACCC | TCAGGAGCCTCATACTTTGG | TTCCAGCAGGATTTCTGGTT | C | T |
| Control_Region_1 | CTCAGTGTGGTTAGAGTTGG | CTGTTCCATTTTGCAACGCC | AGAAAAGACAGATTGCAC | T | C |
| Control_Region_2 | CTTATCAATTACTTTCCTCCC | GGTTTTTCACAGAGGTTTAAG | CCTCCCATTTTAAATTCAATTTAT | G | T |
| Control_Region_3 | TGGGATCTTTGCCATTAGGG | TACCTGTGTCAGAACAAATC | AGCTTTGCCATTAGGGACCATC | T | C |
| Control_Region_4 | CTCTTACAGCCAACTAGCTC | GAGAGCAAATGCACTTACCC | AAAGCCAACTAGCTCATGATCGCTC | C | T |
| VIPR2-L | TCCACCTTCCTGATCATGTG | GGGAAGATTCTCCTAATGGG | CGGACCTCTAATTCCTTCACCTTC | C | A |
| VIPR2-R | ATTTGGGAAAGCAAGTGGGC | AGTCACTCATTGCCTGGAAG | GGGCAGAGTGGGCAGTGAC | C | T |

All patents, patent applications, publications, product descriptions and protocols, cited in this specification are hereby incorporated by reference in their entirety. In case of a conflict in terminology, the present disclosure controls.

While it will be apparent that the disclosure herein described is well calculated to achieve the benefits and advantages set forth above, the present disclosure is not to be limited in scope by the specific embodiments described herein. It will be appreciated that the disclosure is susceptible to modification, variation and change without departing from the spirit thereof.

1. A method for diagnosing schizophrenia in a subject comprising detecting a VIPR2 CNV in a sample from the subject, wherein detecting the VIPR2 CNV indicates a diagnosis of schizophrenia.

2. The method of claim 1, wherein the subject is a human, and wherein the VIPR2 CNV comprises nucleic acid in region 7q36 of human chromosome 7.

3. The method of claim 2, wherein the VIPR2 CNV comprises a region of nucleic acid that is within 89 kb of the transcriptional start site of a VIPR2 gene

4. The method of claim 2, wherein the nucleic acid in region 7q36 comprises nucleic acid in region 158,448,321-158,810,016 of human chromosome 7.

5. The method of claim 1, wherein the VIPR2 CNV comprises nucleic acid of exon 3 or exon 4 of a VIPR2 gene.

6. A method for diagnosing schizophrenia in a subject comprising:
(i) determining VIPR2 expression level in a first sample from the subject;
(ii) determining VIPR2 expression level in a second sample from a second subject that does not have schizophrenia; and
(iii) comparing the level of VIPR2 expression in the first sample with the level of VIPR2 expression in the second sample,
wherein an increased level of VIPR2 expression in the first sample compared to the level of VIPR2 expression in the second sample indicates a diagnosis of schizophrenia.

7. The method of claim 6, wherein the VIPR2 expression is selected from the group consisting of VIPR2 mRNA expression and VIPR2 protein expression.

8. A method for diagnosing schizophrenia in a subject comprising:
determining, in a sample from the subject, the change in the level of cyclic-AMP in response to one or more agent selected from the group consisting of vasoactive intestinal peptide, BAY 55-9837, and another agonist of VIPR2;
(ii) comparing the change in cyclic-AMP level detected in (i) with the change in cyclic-AMP level, in response to one or more of the agents, observed in one or more samples from a control subject that does not have schizophrenia; and
wherein an increased change in the level of cyclic-AMP in the sample from the subject compared to the change observed in one or more control subject samples indicates a diagnosis of schizophrenia.

9. The method of claim 1, wherein the detecting is nucleic acid detection and the nucleic acid detection is an assay selected from the group consisting of polymerase chain reaction (PCR), quantitative PCR, nucleic acid sequencing, nucleic acid microarray analysis, and fluorescence in situ hybridization.

10. The method of claim 9 where the assay is nucleic acid microarray analysis, where the microarray is used to detect the presence of single nucleotide polymorphisms and copy number variation.

11. The method of claim 9 where the assay is fluorescence in situ hybridization, and is used to detect copy number variation.

12. The method of claim 6, wherein the detecting is immunodetection and the immunodetection is selected from the group consisting of ELISA, Western blot, and radioimmunoassay (RIA).

13. The method of claim 12, wherein the immunologic detection comprises detection with an antibody selected from the group consisting of a polyclonal antibody and a monoclonal antibody.

14. A kit for detecting a VIPR2 CNV or VIPR2 expression level in a sample, wherein the kit comprises a plurality of oligonucleotide primers, each of which is capable of specifically hybridizing to a nucleic acid associated with a VIPR2 CNV or VIPR2 expression product.

15. A kit according to claim 14, wherein each of the plurality of oligonucleotide primers is immobilized on a solid surface or support.

16. A kit according to claim 14, wherein each oligonucleotide primer is immobilized at a known position on the solid surface or support.

17. A method for treating a subject comprising:
(i) testing a sample from the subject for the presence of a VIPR2 CNV; and
(ii) administering an agent to the subject if the VIPR2 CNV is present,
wherein the agent is administered in an amount effective to reduce the expression of a VIPR2 gene or function of a VIPR2 protein.

18. The method of claim 17, wherein the agent is selected from the group consisting of a VIPR2 protein antagonist, VIPR2 antisense molecule, VIPR2 RNAi molecule, and VIPR2 siRNA molecule.

19. The method of claim 1, wherein the VIPR2 CNV is detected in a human, and is associated with a second disorder.

20. The method of claim 19, wherein the second disorder is autism.


SEQUENCE LISTING (24 sequences)

SEQ ID NO: 1 - DNA, 21 bp, Artificial sequence
  Synthetic oligonucleotide
cagattttattaggacaaagc

SEQ ID NO: 2 - DNA, 20 bp, Artificial sequence
  Synthetic oligonucleotide
gggtaatttttctcaattcc

SEQ ID NO: 3 - DNA, 19 bp, Artificial sequence
  Synthetic oligonucleotide
agtaggacaaagcagaaca

SEQ ID NO: 4 - DNA, 20 bp, Artificial sequence
  Synthetic oligonucleotide
gcatctacttccagttaccc

SEQ ID NO: 5 - DNA, 20 bp, Artificial sequence
  Synthetic oligonucleotide
tcaggagcctcatactttgg

SEQ ID NO: 6 - DNA, 20 bp, Artificial sequence
  Synthetic oligonucleotide
ttccagcaggatttctggtt

SEQ ID NO: 7 - DNA, 20 bp, Artificial sequence
  Synthetic oligonucleotide
ctcagtgtggttagagttgg

SEQ ID NO: 8 - DNA, 20 bp, Artificial sequence
  Synthetic oligonucleotide
ctgttccattttgcaacgcc

SEQ ID NO: 9 - DNA, 18 bp, Artificial sequence
  Synthetic oligonucleotide
agaaaagacagattgcac

SEQ ID NO: 10 - DNA, 21 bp, Artificial sequence
  Synthetic oligonucleotide
cttatcaattactttcctccc

SEQ ID NO: 11 - DNA, 21 bp, Artificial sequence
  Synthetic oligonucleotide
ggtttttcacagaggtttaag

SEQ ID NO: 12 - DNA, 24 bp, Artificial sequence
  Synthetic oligonucleotide
cctcccattttaaattcaatttat

SEQ ID NO: 13 - DNA, 20 bp, Artificial sequence
  Synthetic oligonucleotide
tgggatctttgccattaggg

SEQ ID NO: 14 - DNA, 20 bp, Artificial sequence
  Synthetic oligonucleotide
tacctgtgtcagaacaaatc

SEQ ID NO: 15 - DNA, 22 bp, Artificial sequence
  Synthetic oligonucleotide
agctttgccattagggaccatc

SEQ ID NO: 16 - DNA, 20 bp, Artificial sequence
  Synthetic oligonucleotide
ctcttacagccaactagctc

SEQ ID NO: 17 - DNA, 20 bp, Artificial sequence
  Synthetic oligonucleotide
gagagcaaatgcacttaccc

SEQ ID NO: 18 - DNA, 25 bp, Artificial sequence
  Synthetic oligonucleotide
aaagccaactagctcatgatcgctc

SEQ ID NO: 19 - DNA, 20 bp, Artificial sequence
  Synthetic oligonucleotide
tccaccttcctgatcatgtg

SEQ ID NO: 20 - DNA, 20 bp, Artificial sequence
  Synthetic oligonucleotide
gggaagattctcctaatggg

SEQ ID NO: 21 - DNA, 24 bp, Artificial sequence
  Synthetic oligonucleotide
cggacctctaattccttcaccttc

SEQ ID NO: 22 - DNA, 20 bp, Artificial sequence
  Synthetic oligonucleotide
atttgggaaagcaagtgggc

SEQ ID NO: 23 - DNA, 20 bp, Artificial sequence
  Synthetic oligonucleotide
agtcactcattgcctggaag

SEQ ID NO: 24 - DNA, 19 bp, Artificial sequence
  Synthetic oligonucleotide
gggcagagtgggcagtgac